(19) United States
(12) Patent Application Publication
(10) Pub. No.: US 2024/0216100 A1
(43) Pub. Date: Jul. 4, 2024

(54) PACKAGING SYSTEM FOR A MEDICAL TUBE SET

(71) Applicant: Xenios AG, Heilbronn (DE)

(72) Inventors: Hans-Günther Schon, Alsheim (DE); Alice Watmann, Benningen (DE); Detlef Günther, Filderstadt (DE)

(21) Appl. No.: 18/042,745

(22) PCT Filed: Aug. 23, 2021
(86) PCT No.: PCT/EP2021/073258

§ 371 Date: Feb. 23, 2023

(30) Foreign Application Priority Data
Aug. 25, 2020 (DE) 10 2020 005 197.9

(51) Int. Cl. A61B 50/20; A61B 50/33

(52) U.S. Cl. CPC A61B 50/20; A61B 50/33

(57) ABSTRACT

The present disclosure relates to a packaging system for a medical tubing set for extracorporeal therapy, wherein the packaging system includes a tray, which is configured to receive an inlay as well as to reversibly fasten the inlay in the tray, and an inlay with one or more recesses for receiving the tubing set, wherein a cavity is formed between the tray and the inlay. For example, such a packaging system can have a modular design and allows the medical tubing set to be unpacked in the order of use. The disclosure also relates to the use of such a packaging system for packaging a medical tubing set as well as a method for packaging a medical tubing set.

### CROSS-REFERENCE TO RELATED APPLICATIONS

The present application is the national stage entry of International Application No. PCT/EP2021/073258, Aug. 23, 2021, and claims priority to Application No. DE 102020005197.9, filed in the Federal Republic of Germany on Aug. 25, 2020, the disclosure of which are expressly incorporated herein by reference thereto.

### TECHNICAL FIELD

The present disclosure relates to packaging systems for medical tubing sets, e.g., for extracorporeal therapies. The disclosure also relates to the use of such a packaging system for packaging a medical tubing set and a method for packaging a medical tubing set.

### BACKGROUND

Medical tubing sets are used in a variety of ways in the care of patients. For example, medical tubes can be used to conduct liquids (such as blood, saline and/or medicaments) or gases (e.g., oxygen). Medical tubing sets usually include other items in addition to the tubing that are used together with the medical tubing. For this reason, medical tubing sets are usually assembled according to the specific application. For example, tubing sets for extracorporeal blood treatment (e.g., for removing carbon dioxide from the blood or supplying the blood with oxygen using oxygenators) usually contain tubing for blood conduction, but also gas-carrying tubing (e.g., for oxygen transport). Often medical tubing sets are delivered with the devices intended for the application (e.g., an oxygenator).

The sterility of medical devices or products is of great importance for medical applications. It must therefore be ensured that the sterility of a medical product, such as a medical tubing set, is guaranteed from the time of manufacture to its use in the context of a treatment, e.g., the extracorporeal therapy of a patient. Therefore, the demands on the packaging of medical products are particularly high, as a sterile barrier to the environment must be ensured, which is maintained during realistic storage and transport conditions. Furthermore, the packaging must ensure that the medical product is not damaged during transport.

With conventional packaging for medical products, after unpacking, the medical products usually have to be transported individually to the place of use, e.g., to the therapy area such as an intensive care unit. During this process, the medical products are exposed to the risk of contamination through contact. In addition, there is a risk of damage to individual product components by unprotected, suspended assemblies during transport in the controlled zone (e.g., OR). In addition, hook and loop tape is often used in the packaging of sets, with which the product cannot be fixed in a standardized way, i.e., by means of defined reproducible tightening torques, such that transport damage can occur and long set-up times are required for the assembly. In addition, conventional packaging for medical products often requires a complicated and less usable removal of the products from the packaging.

Against this background, it is an aspect of the present disclosure to overcome the disadvantages of the prior art and to provide an improved packaging system for a medical tubing set. In particular, the safety and functionality of the packaged products during transport can be guaranteed by appropriate storage. In addition, the sterility of the medical products, especially of medical tubes, can be maintained from the packaging to their medical application. In addition, the modular design of the packaging system enables a defined reproducible and user-friendly removal of the medical products essentially in the order of their application. The modular design also allows the packaging system to be easily adapted to different medical tubing sets, e.g. with different or differently dimensioned components, wherein only one respective (or a few) module(s) of the packaging system have to be adapted in each case.

### SUMMARY

In the following, terms such as “approx.”, “approximately”, “about” or “essentially” in relation to numerical specifications generally mean±10% (e.g., ±7%, ±5%, ±2%, ±1%), unless these terms are defined differently for a specific case.

Technical terms usually have their usual, technical meaning, unless they are defined differently herein.

The present disclosure provides a packaging system for a medical tubing set for extracorporeal therapy, wherein the packaging system includes the following components:
a tray configured or adapted to receive an inlay and to reversibly fix the inlay in the tray, and
an inlay with one or more recesses for receiving (components of) the tubing set, wherein a cavity is formed between the tray and the inlay.

The packaging system described herein is particularly designed in a modular manner, such that a simple adaptation of the packaging system to different medical tubing sets, e.g., with different or differently dimensioned components, is enabled, wherein usually only one (or few) respective module(s) of the packaging system must be adapted. Thus, trays can be advantageously of the same size in such a packaging system, even if they are used for packaging of different tubing sets (having different sizes or different circumferences). For example, the inlay for the different tubing sets can be configured and/or formed differently. Thus, the packaging system described herein allows for great flexibility with regard to the packaging of different tubing sets, since only one or a few components of the packaging system (e.g., the inlay—but not the other components of the packaging system) have to be adapted to the different tubing sets.

In addition, the modular design of the packaging system enables a defined, reproducible and user-friendly removal of the tubing set essentially in the order of their application.

In particular, the tubing set can be packaged completely on the inlay. Since the inlay can be reversibly fixed or fastened in the tray, it is easy to pack (and unpack) the tubing set on the inlay outside the tray. The tubing set packed on the inlay can then be placed in the tray in a further step (with the inlay) and be fixed. During unpacking, the reversible fastening of the inlay can also be detached or released so that the inlay with the (packed) tubing set can be removed from the tray and the tubing set (outside the tray) can then be unpacked from the inlay. This enables easy transport of the tubing set (packed on the inlay) to sterile areas (application areas) where a possibly externally contaminated tray is undesirable. The various components of the tubing set do not have to be transported individually to particularly sterile areas in a complicated manner, but can be easily transported to the area together with the (sterile) inlay.

An exemplary medical tubing set, which can be packed in the packaging system described herein, can include a tube for blood conduction, a tube for gas conduction, and optionally an oxygenator and/or a pump head. Furthermore, various additional items that are usually used together with the medical tubing set, such as tubing clips, a syringe, cable ties, etc., can also be packaged in the packaging system described herein (and therefore included for supply).

The cavity formed between the tray and the inlay (especially when the inlay is fixed in the tray) serves to safely transport sensitive components of the tubing set (e.g. an oxygenator and/or a pump head), since the cavity between the inlay and the tray can absorb vibrations to protect the tubing set.

Since the packaging system is used to package a medical tubing set, as is the case in the medical sector, e.g., in an intensive care unit, the packaging system (and the tubing set packed in it) can be sterile, e.g., inside the tray. For example, the components of the packaging system (and the tubing set) are sterilized before and/or during the packaging of the tubing set in the packaging system. Then the tray (with the tubing set packed therein) is closed, e.g., sealed, e.g. with a foil. The material of the sealing (foil) is, e.g., suitable for both sealing and sterilization, e.g., Tyvek® (PE-HD). The sterility of the packaging system inside the tray (and the tubing set packed therein) is hence maintained even in the event of external contamination (e.g. during transport and/or storage), even if the tray may lose its sterility externally.

The inlay of the packaging system includes one or more recesses to accommodate or receive (components of) the tubing set. For example, the inlay may have recesses for receiving one or more tubes. In addition, the inlay may have one or more receiving section(s), e.g. for receiving an oxygenator (and/or pump head). In contrast to the recesses, receptacle or receiving sections may include both recesses (in the inlay) and elevations or protrusions (extending from the inlay).

The shape of the recess(es) and the receiving section(s) can be advantageously adapted to the shape of the object (component of the tubing set) to be received in/on it. For example, the shape of the recess or the receiving section may correspond to (a subsection of) the external shape of the object to be received. Thus, objects (tubing set components) can be received/arranged in/at/on the inlay by the recess(es) or receiving section(s), for example, in an essentially form-fitting manner.

However, the shape of the recess(es) does not have to correspond to the external shape of the object to be received. Hence, the inlay may have one or more recesses which do not correspond to the external shape of the object to be received, but which allow a certain flexibility in accommodating the object. This can be advantageous, for example, when receiving or accommodating tubes to allow easy packing and unpacking. In particular, tubes do not usually have to be fixed over their entire length (at every point) for safe transport. The packaging system can therefore enable objects to be fixed at particular points (e.g. by means of a partition in the case of tubes). For example, recesses provided for tubes can be advantageously configured to be larger than the tube circumference in order to allow a certain amount of play when packing or unpacking the tube and to prevent the tube from kinking. For example, recesses provided for receiving tubes can be configured to accommodate several layers or loops of the tube or several tubes. Round or elliptical recesses or receptacles for tubes are advantageous. In a recess or receiving section foreseen for a tube, an elevation or protrusion may be arranged approximately centrally around which the tube can be wound.

In the inlay several recesses may be present which are separate (separated from each other) or connected to each other (e.g. by channel-like connecting recesses). While separate recesses usually have a completely circumferential inner wall (without cut-outs or interruption), interconnected recesses have lateral cut-outs through which they are connected to each other, e.g. by channel-like connecting recesses. Thus, for example, one and the same tube can be packed in several recesses by packing one or more loops of the tube in a first recess, then passing the tube through a channel-like connecting recess of a second recess and packing one or more loops of the tube in this (second) recess.

The tray usually has a bottom or base and a wall, where the inside of the wall is also called “inner wall” and the outside of the wall is also called “outer wall”. The wall is also referred to herein as the “side wall” or “side walls”.

The size of the tray can be determined by the size of the inlay or by the size of the packed tubing set. An exemplary tray for packaging a medical tubing set with a tube for blood conduction, a tube for gas conduction, an oxygenator, as well as a pump head may have a length of about 50-65 cm, e.g., 55-60 cm, 56-57 cm; a width of about 40-55, e.g., 45-50 cm, about 47 cm; and/or a height of about 12-20 cm, e.g., 14-18 cm, about 16-16.5 cm. The wall thickness of the tray can be about 2−3 mm (e.g., about 2.5 mm).

Trays for different medical tubing sets can be advantageously of the same size, wherein the inlay for different tubing sets may be configured differently (but in such a way that the inlay fits into the “universal” tray). Thus, the packaging system described herein allows great flexibility with regard to the packaging of different tubing sets, since (only) the inlay (but not the other components of the packaging system) must be adapted to the different tubing sets.

The shape of the tray is usually determined by its base area (bottom) and is not limited. However, trays with essentially rectangular base areas are particularly advantageous for storage and transport, as they can be transported and stored in a space-saving manner. An “essentially rectangular” base area is formed by four sides arranged (essentially) at right angles to each other, with the respective opposite sides being (essentially) parallel to each other. The sides may have recesses, protuberances, bendings/curves or similar, as long as the respective (imaginary) baselines of the sides meet the above requirements. The “corners” of the rectangle may be rounded or beveled or tapered.

The tray can be made, e.g., of a plastic such as PET (polyethylene terephthalate) or PETG (PET modified with glycol). The tray can be manufactured in a variety of ways, including thermoforming and 3D printing methods.

The walls of the tray can be substantially essentially vertical to the bottom of the tray. “Substantially vertical” includes deviations from the vertical axis of up to 20 from the bottom or base; e.g., deviations from the vertical axis of up to 15′ from the bottom, up to 10, or up to 5. Thus, the tray can advantageously have (side) walls that are slightly inclined outwards. For example, the walls can be inclined outwards by up to 20′ (e.g., up to 15, up to 10, up to 5′, up to 4′ or 3, or up to 2′ or 1′). In this way, the tray has, by comparison, a base area (at the bottom of the tray) which is smaller than the plane formed by the (essentially horizontal) upper edge of the tray. Such a smaller base area (at the bottom) compared to the plane formed by the upper edge can also be achieved by steps in the side walls of the tray. Steps in the sidewalls of the tray can be in vertical sidewalls or advantageously in slightly outwardly inclined sidewalls. Such trays, where the base area (at the bottom) is smaller than the plane formed by the upper edge, are advantageous, as they can be easily stacked and therefore stored in a space-saving manner.

The upper edge of the tray can be advantageously designed substantially horizontally. For example, the upper edge (compared to the wall thickness of the tray) is widened in the horizontal plane, e.g., by “bending” the side walls of the tray horizontally outwards. In this way, a cover (e.g. a foil) can be easily applied to seal the tray. This way the upper edge of the tray can have a width of approx. 5 to 20 mm, e.g., 7-15 mm, 10-12 mm, or 10.5-11 mm.

In some examples, the tray and/or inlay are adapted, such that all components of a tubing set, as well as all potential further items to be packaged, may be (entirely) packaged on (i.e. on top of) the inlay. In this manner, all components or items to be packaged benefit from the cavity (for shock absorption) between the tray and inlay. In this manner, all packaged components and items may furthermore be simply taken together with the inlay out of the tray (and potentially be transported further).

The shape (contour) and/or size of the inlay may advantageously correspond to the shape and/or size (of the interior space) of the tray. In other words, the inlay may advantageously extend essentially over the entire area or portion (e.g. bottom) of the tray. In particular, the tray (interior) may have a length and width, which does not respectively exceed the length and width of the inlay by more than 3 cm, e.g., not more than 2 cm, not more than 1 cm, or not more than 0.5 cm. Advantageously, the inlay (length and width) is only marginally smaller than the tray. This enables that the inlay advantageously extends essentially over the entire area (e.g. bottom) of the tray (hence enabling a space-saving packaging), but may also be easily placed into and be removed from the tray. In this regard it is particularly advantageous, if the inlay may be placed on an substantially horizontal support surface at the inner wall of the tray.

The tray may have an substantially horizontal support surface advantageously at its inner wall (for supporting the inlay). The supporting surface can extend (over all inner walls of the tray) essentially circumferentially around the tray. Alternatively, the support surface on the inner wall of the tray can e.g. be realized by one or more (horizontal) support points on each of the four inner wall sides of the tray. The support surface can be achieved, for example, by a step in the (inner) wall. Such a step in the (inner) wall of the tray has the advantage that the tray is (better) stackable. In particular, the step maintains a distance which counteracts vacuum formation during unstacking. Alternatively, the support surface can also be formed by a projection or protrusion. The support surface can have a width of approx. 3 to 25 mm, for example.

On this support surface the inlay, especially the outer edge on the bottom side of the inlay, can be placed (essentially in a form-fitting manner) such that horizontal sliding of the inlay in the tray is prevented or at least limited. The support surface is advantageously arranged in the lower half of the wall height of the tray, e.g., in the lower third of the wall height. For example, the support surface can be arranged not (lower than) in the lower fifth, e.g., (lower than) in the lower quarter of the wall height of the tray. In this way, sufficient space is ensured above the support surface (inlay) for the arrangement of the tubing set and below the support surface (below the inlay) sufficient space remains for the arrangement of the recesses in the inlay without the (undersides of the) recesses touching the bottom of the tray. When packing the tubing set, the inlay can be placed on the support surface of the inner wall of the tray so that the underside of the outer edge of the inlay is at least partially in contact with the support surface of the tray. In this way, the inlay can be stabilized in vertical direction in the tray. Advantageously, the tray is dimensioned in such a way that the inlay can rest simultaneously on support surfaces on all inner wall sides (e.g. on all four inner wall sides in case of a rectangular basic shape) and the inner walls of the tray limit or essentially prevent a horizontal movement of the inlay. In this way, an (unintentional) horizontal movement (such as slipping or sliding) of the inlay in the tray is (at least partially) prevented or limited. According to some embodiments, the inlay is not in contact with the bottom of the tray.

It is particularly advantageous, if the support surface for the inlay includes an arrow-shaped recess or elevation or protrusion at one or more positions in the tray. In accordance, the inlay advantageously also includes a corresponding arrow-shaped recess or elevation or protrusion (essentially in a positive locking manner) at a corresponding position. In this manner the arrow-shaped recess or elevation or protrusion in the inlay may be arranged (essentially with exact fitting) onto the corresponding arrow-shaped recess or elevation or protrusion in the support surface of the tray. Thereby, the stability of the inlay in the tray may be further increased. For this purpose, the support surface for the inlay within the tray may be advantageously widened at one or more sites. Particularly advantageous, the arrangement of the arrow-shaped recess or elevation or protrusion is located at a corner of the tray or inlay, respectively.

Thereby, a corner of the inlay may advantageously include an arrow-shaped recess (or an arrow-shaped elevation or protrusion). For this purpose, the tray may include a corresponding essentially positive locking arrow-shaped recess (or an arrow-shaped elevation or protrusion) in the corresponding corner. The arrowhead of the arrow-shaped recess (or elevation or protrusion) of the inlay or of the tray can point in the diagonal direction towards the corner (edge) of the inlay or tray. The tip or apex of the arrowhead may be rounded or flattened or tapered (i.e. without a true “barb”). The rear, wide end of the arrowhead is arranged essentially perpendicular to the diagonal orientation of the arrow. Both of the edges of the recess (or elevation or protrusion) forming the rear, wide end of the arrowhead are hence arranged essentially perpendicular to the diagonal orientation of the arrow. In this manner the fixation of the inlay in the tray may be further improved.

For this purpose it is particularly advantageous, if the inlay and the tray respectively include two (between the inlay and the tray correspondingly positively locking) arrow-shaped recesses (or elevations or protrusion), e.g., in diagonally opposing corners of the inlay and tray. The diagonally opposing corners of the inlay or tray with an arrow-shaped recess may furthermore be advantageously adapted for the application of an fastening means (e.g. as described in the following)—for example by corresponding seatings or receptacles for a latch or locking mechanism by the fastening means in the tray and by corresponding openings for the fastening means in the inlay. It is particularly advantageous, if all four corners of the inlay and tray include respective (between the inlay and the tray correspondingly positively locking) arrow-shaped recesses (or elevations or protrusion). In this manner, it is avoided that the inlay slides or moves within the tray, even in the case of a drop or fall (e.g. during transport).

The inlay can be reversibly fixed or fastened in the tray. The reversible fastening of the inlay in the tray can be performed in different ways, e.g. by means of a locking mechanism or device (e.g. screw cap or twist lock), by clicking, pushing, or tightening (e.g. with screws as fastening means). The inlay may be inserted or clicked into the tray without any further fastening means. For this purpose, the tray may be provided with corresponding recesses, openings, cut-outs, projections or grooves that allow the inlay to be received in a reversible manner. The inlay can also have recesses, openings, cut-outs, projections or grooves (matching those in the tray) for reversible fastening in the tray. The reversible fastening, e.g. the fastening means, may be advantageously combined with the above-described arrow-shaped recesses (or elevations or protrusion) in the inlay or tray, respectively.

In some examples, the packaging system described herein includes a fastening means for reversible fastening of the inlay in the tray. The fastening means can be attached to the inlay, for example. The fastening means may be fixed or mounted movably (e.g. rotatable) on the inlay. The connection of the fastening means to the inlay may be reversible or irreversible. For example, the inlay may have openings to allow the fastening means to pass through the inlay and/or to attach it to the inlay in a rotatable manner. The fastening means can fasten the inlay reversibly in the tray, for example by means of a locking mechanism or device (e.g. by means of a twist lock).

The fastening means can advantageously have an upper part (e.g. a handle of the fastening means) and a lower part (e.g. a base of the fastening means). The upper part (handle) may have a gripping section and an extension for connection to the base, with the extension or projection passing through an opening in the inlay. In this way, the gripping portion of the upper part (handle) and the lower part (base) of the fastening means (when connected) can be arranged on opposite sides of the inlay. The lower part (base) may be arranged substantially below the inlay (and may optionally extend upwards into or through the opening in the inlay). The upper part (handle) of the fastening means may be arranged substantially above the inlay (and may optionally extend downwards into or through the opening of the inlay). A small (partial) portion of the lower part (base) of the fastening means may also be arranged above the inlay and/or a small (partial) portion of the upper part (handle) of the fastening means may also be arranged below the inlay, e.g. to connect both parts of the fastening means with each other.

For example, the fastening means, in particular the upper part (handle), may be connected to the inlay (or be applied thereto). In particular, the handle may be adapted to be used for removal of the inlay or for its transport (with the tubing set and potentially further packaged items) in addition to the operation or function of the fastening means. In some examples, the handle is hence connected to the inlay and adapted such that it may serve for removal and/or transport of the inlay, in particular with the components of the tubing set being packaged thereon, as well as all potentially further packaged items.

For example, the upper part (handle) of the fastening means may have a gripping section and an extension for connecting to the lower part (base) of the fastening means. The extension of the upper part (handle) of the fastening means can be passed through (or into) an opening of the inlay, in particular to connect the upper part (handle) of the fastening means on the top of the inlay with the lower part (base) of the fastening means on the bottom of the inlay. For this purpose, the lower part (base) of the fastening means can advantageously have a (precisely fitting) receptacle for the extension of the upper part of the fastening means. By inserting the extension into the receptacle or seat, a suitable connection of the upper and lower part of the fastening means can be achieved.

Such a connection of the upper part (handle) with the lower part (base) of the fastening means generally allows movements actuated on the upper part (handle) of the fastening means, in particular on its gripping section—such as a rotary movement—to be transmitted to the lower part (base) of the fastening means. If, for example, the user performs a turning or rotating movement on the gripping section, this will also turn or rotate the base of the fastening means (located below the inlay).

The upper part (handle) of the fastening means allows, for example, the user to operate the fastening means. This enables the user to use the handle (gripping section) to fasten or detach the inlay in the tray (e.g. by manually operating the fastening means). The lower part (base) of the fastening means can, for example, be used for locking, e.g. in combination with projections of the inner wall of the tray. In some examples, the lower part (base) of the fastening means has one or more wings.

In some examples, the fastening means (in the inlay) is rotatable. One (or more) wings of the base of the fastening means can be positioned in a receptacle or beneath projections of the (inner) wall of the tray by rotating the fastening means (e.g. by the user on the handle). This can substantially prevent vertical movement of the inlay out of the tray.

For this purpose, the tray can (in the area close to the floor, especially in the lower third or quarter of the wall height) have a receptacle or seat for locking by the fastening element (fastening means). Such a receptacle can be formed by one or more appropriately dimensioned projection(s) in the (inner) wall of the tray. In other words, the tray may have (near to its bottom) one or more projections or protrusions on its inner wall beneath which a wing of the base of the fastening means can be positioned.

Advantageously, the tray may include e.g. two seatings or receptacles for a locking mechanism by means of two fastening means, in particular with (e.g. two respective) projections. Such a packaging system thus includes, in addition to the inlay and tray, two fastening means for reversibly fastening the inlay in the tray. Here, the seatings or receptacles for both of the fastening means (and potentially the corresponding openings in the inlay) are possibly spaced apart from each other in such manner that the distance between both of the seatings or receptacles in the tray is larger than the distance from each of the seatings or receptacles or receptacles to the respective closest edge of the tray. In particular, the distance between both of the seatings or receptacles in the tray are at least double, e.g., at least four times, at least six times, at least eight times, or at least ten times as large as the distance of each of the seatings or receptacles to the respective closest edge of the tray. If the basic shape of the tray is rectangular, two receptacles with (e.g. two) projections each may be arranged in the diagonally opposite corners of the tray. Exactly two receptacles can be provided for fastening the inlay in the tray. However, it is also possible to have, for example, four receptacles for fastening the inlay in the tray, e.g. one receptacle in each corner or one receptacle on each inner wall (side surface) of the tray. The receptacle for fastening the inlay in the tray can, for example, have (exactly) one projection or two, in particular opposite, projections, which is/are dimensioned in such a way that one wing of the base of the fastening means arranged below the inlay at least partially engages beneath the projection(s), when the fastening means is moved (e.g. rotated). This means that the inlay can no longer (or only to a limited extent) be moved upwards and removal of the inlay from the tray can be prevented. If the fastening means is moved in the opposite direction (e.g. turned in the opposite direction), the wing in the lower part (e.g. the base) of the fastening means will no longer engage beneath the projection(s). This allows the inlay to be moved (again) in a vertical direction and the inlay can thus be easily and reversibly removed from or inserted in the tray.

In this way, depending on the position of the wing of the base of the fastening means (in relation to the projections of the inner wall of the tray), the projections on the inner wall of the tray cause the inlay to be fixed or detached in the tray. When the fastening means is rotated, the wing can be moved in relation to the one or more projections of the inner wall of the tray in such a way that the inlay is fastened/fixed or detached in the tray.

The interaction of the projection(s) of the (inner) wall of the tray with the wing in the lower part (e.g. the base) of the fastening means therefore enables locking by moving the wing relative to the projection(s). When the fastening means for reversible fixing of the inlay in the tray is rotated, it can e.g. be rotated by about 90 (especially with two opposite projections) to fasten or release the inlay in the tray. However, depending on the dimensioning of the wing in comparison to the projection, smaller or larger rotation angles can also be used for locking or releasing the lock.

For example, the user can rotate a wing (the base) of the fastening means by turning the gripping section (the handle) so that it is positioned beneath a projection of the (inner) wall (and thus fasten the inlay in the tray). Similarly, a (further or opposite) rotation of the gripping section can release the fastening of the inlay in the tray, if the wing of the base of the fastening means is no longer positioned beneath a projection of the (inner) wall of the tray. Such a locking mechanism can be configured in such a way that it locks in the best possible position (e.g. with the greatest possible overlap of wing and projection; e.g. after turning by approx. 90′). The outer side edges of the wing can have a rounded or chamfered shape, e.g. to facilitate the turning movement or to enable or facilitate the locking mechanism to engage.

For example, according to an embodiment, a combination of
(i) a projection of the (inner) wall of the tray with a wing in the lower part (e.g. the base) of the fastening means (limiting or preventing vertical movement upwards (out of the tray); and
(ii) (a) a support surface of the tray (with the edge area of the inlay resting on it), or
   (b) accordingly dimensioned recesses in the tray (which limit or prevent vertical movement of the inlay downwards (into the tray)

advantageously minimize or essentially prevent movement of the inlay in vertical direction altogether.

The gripping section of the upper part (e.g. a handle) of the fastening means can have different shapes. The gripping section can be adapted to the movement to be performed for the fastening. For example, gripping sections of a particular width, which can be easily operated with one hand, are advantageous for manual turning movements.

In order to enable an as easy to operate and easy to reach gripping section, the upper part of the fastening means, which is located above the inlay, can be dimensioned such that the gripping section is arranged in an upper section of the tray, e.g., as close as possible below the upper edge of the tray. For example, the upper part of the fastening means above the inlay may be dimensioned such that the top of the handle of the fastening means does not protrude above the top edge of the tray (so that when the inlay is attached, the tray can be sealed without restriction). Alternatively, the gripping section can be provided in a middle or lower section of the tray.

The fastening means, in particular its upper part (handle), can contain a hook in some embodiments, which can be released (e.g. broken out) from the fastening means if required.

Thus, in a further aspect, the packaging system also provides a fastening means for reversibly fastening an inlay in a tray, including a handle and a base, the handle having a gripping portion and an extension for connection to the base, which can be passed through an opening in the inlay, wherein a hook is integrated in the handle, which can be released from the handle. The detailed description of the fastening means in the context of the packaging system is therefore also applicable to the fastening means and vice versa.

The hook in the upper part (handle) of the fastening means may have a holder for fastening the hook to a rod or rail, for example. Rails or rods found in the medical therapy area (e.g. on the console where also the oxygenator and/or an electric motor for driving the blood pump of the tubing set can be placed during operation on the patient) often have dimensions according to DIN standards, so that a correspondingly adapted holder, and thus the hook, can be used widely in this area. For example, the holder can be configured in such a way that the hook can be easily clipped onto a rod or rail.

In particular, neither the fastening means nor the hook will be damaged and/or neither the functionality of the fastening means nor the functionality of the hook will be impaired by releasing the hook from the fastening means. For this purpose, the hook can only be integrated into the fastening means by means of a few (e.g. two or three) connecting bridges that are as thin as possible. The connecting bridges can, for example, have predetermined breaking points, e.g. by making certain sections of the connecting bridges particularly thin (thinner than the remaining connecting bridge). The upper part of the fastening means with the integrated hook can be advantageously made in one piece, so that the upper part with the integrated hook can be produced in one and the same operation step (and no additional operation steps are required to integrate the hook into the fastening means). Therefore, the hook is usually made of the same material as the fastening means or its upper part, respectively.

The material of the fastening means is not restricted as long as the functionality of the fastening means is guaranteed and the material can be sterilized. The upper part (handle) and the lower part (base) of the fastening means may be made of the same material or of different materials. Advantageous materials are plastics, such as polypropylene and PBT (polybutylene terephthalate). PBT is an example of a material for the lower part of the fastening means (base) or for the entire fastening means, as the fibers integrated in the material advantageously increase the stability. The upper part of the fastening means (handle) can also be advantageously made of polypropylene to make it easier to break the hook, if present, out of the handle.

The fastening means can also be used to transport the inlay (outside the tray)—and thus the objects packed on it/therein as well as other elements of the packaging system that are optionally present on the inlay—e.g. in intensive care units. Such a transport of the inlay at the fastening means (with the packed objects) can be performed e.g. during packing, if the tubing system is advantageously packed on or in the inlay outside of the tray, so that afterwards only the inlay has to be put into the tray and the inlay with the fastening means attached thereto has to be fixed in the tray. When unpacking (e.g. in the intensive care unit), it is also possible to first release or detach the fastening of the inlay in the tray and then remove the inlay with the objects of the tray on it and take it to a destination site for (further) unpacking. The inlay with one or more fastening means is therefore also suitable for safe transport of the tubing set (without tray), e.g. within sterile areas. In this manner, contact with the tubing set (and thus possible contamination) during transport (e.g. to the destination site in the intensive care unit) can be avoided. Accordingly, the inlay is advantageously adapted such that all components of the tubing set, as well as all potential further items to be packaged, may be packaged (entirely) on (i.e. on top of) the inlay.

Furthermore, the packaging system described herein may have one or more covers for one or more recess(es) or one or more receiving section(s) in the inlay. The cover can cover the recess or the receiving section, in particular with the tubing set component(s) packed therein/on top of it, for example, in an essentially form-fit manner. Accordingly, distinct compartments in the packaging system can be created in a modular way. This enables both an orderly packaging (so that particular tubing set components do not come into contact with each other during transport and therefore cannot damage each other); a clearly presented arrangement of the packaged tubing set components and unpacking/removal of the tubing set in the order of use. Thus, the packaged items packed on (above) a cover are visible at first and are therefore usually removed from the packaging system first during unpacking, whereas items packed under a cover are unpacked later (after removal of the cover). This allows the user to intuitively remove the packaged tubing set step by step in the order of use. The cover itself may have one or more recess(es) or receptacle section(s) to accommodate components of the tubing set or additional items that can be used in conjunction with the medical tubing set, such as tubing clips. In this way, components of the tubing set (or additional items) can be placed on top of each other in the packaging system (separated from each other by the cover) without damaging each other. These recess(es) or receiving section(s) can, for example, be designed essentially for form-fitting receiving or as form-fitting receptacles, for example by essentially reflecting the shape of the object to be received.

For example, the packaging system may include a (essentially positive locking) cover for a recess in the inlay, which may serve to cover an item, e.g. a tube, being supported in a recess of the inlay. Such a cover may by itself include one or more recess(es) for receiving or arranging in an essentially positive locking manner components of the tubing set or of additional items, that may be used in accordance with the medical tubing set, such as for receiving or accommodating of tube clamps (in an essentially positive locking manner).

A further cover may e.g. be adapted for covering the first cover. As such, the first cover may serve to cover an item, e.g. a tube, being supported in a recess of the inlay and at the same time components of the tubing set or additional items may be arranged on the first cover, as described above. The further cover then serves to cover the components or items being supported or mounted on the first cover. In this manner, multiple covers may be arranged (stacked) on top of each other in a space-saving manner, wherein advantageously components of the tubing set or additional items may be separated from each other and be packaged in a clearly presented manner. The cover arranged at the top may include e.g. a guide for a fixation means (e.g. a strap), which serves for fixation of components of the tubing set (or further items to be packaged) on the inlay and/or on one or more covers. As such, the (top-most) cover—as well as any potential cover(s) arranged beneath—and items being supported therein, may be fixed.

Also the further cover may by itself include one or more recess(es) or elevations or protrusions for receiving or arranging components of the tubing set or additional items, which may be used in accordance with the medical tubing set, in an essentially positive locking (i.e. form-fitting) manner. For example, the (further or top) cover may include elevations or protrusions for receiving/arranging a syringe. The guide may also be arranged on an (e.g. essentially circular or elliptical) elevation or protrusion, which by itself enables the receiving or arrangement of a tube (coiled around the elevation). The recesses or elevations for receiving or accommodating components of the tubing set or additional items are advantageously arranged such that items arranged therein may also be additionally fixed by the fixation means guided through the guide.

Furthermore, in some embodiments of the packaging system described herein, one or more of the recesses in the inlay may have a lateral recess or cut-out at least on one side of the recess. Such a recess is not completely closed off laterally, even if it is (positively) covered with a cover as described above. In this way, for example, one or more tubes can be inserted in or removed from the side of the recess. In the lateral cut-out of the recess, a partition can be arranged (e.g., form-fitting) through which an object (component of the tubing set, e.g., a tube) can be passed or guided, e.g., non-positively or form-fittingly (e.g., non-positively, force-fit or by interference fit). Thereby, the recess with cover and partition can form a particularly protected compartment (covered inner space or interior of the recess), which is closed, for example, except for the objects (such as tubes) that are guided through or out of the partition. Such a compartment is particularly protected against contamination.

One or more recesses in the inlay may be configured to form particularly protected compartments in the packaging system by means of a cover and suitable sealing means (e.g. a partition). Such particularly protected compartments can, for example, be used to accommodate open ends of a (blood-carrying) tube, which must be particularly protected from contamination (e.g. by smallest dust particles). In order to pass or guide a tube into or out of such a particularly protected compartment, one or more partitions can be arranged in lateral cut-outs of the recess.

The packaging system may therefore also include one or more partitions, which may have one or more openings for guiding tubes (as closely fitting as possible) therethrough. In particular, the partition may have an external shape (outline or contour) which can fill a lateral cut-out of a recess (in the inlay) in a form-fitting manner. In this way, by means of partitions in a recess of the inlay with the cover, an, e.g., closed, interior space can be formed, which is particularly protected from contamination and into which one or more tubes (essentially non-positive) can still be guided in or out by means of the openings of the partitions. Furthermore, the partitions can be used to lock the tubes in the packaging system (for which the outer shape design is usually irrelevant).

Generally, exactly one tube can be guided through exactly one opening of a partition. The one or more openings in the partition can be adapted exactly to the tube circumference (tube size). This prevents contamination of the formed compartment (interior of the recess). Thus, a packaging system can include one or more partitions, wherein all openings of the partition are essentially identical (and therefore configured for the same tube size, e.g. a blood-carrying tube). Alternatively, a packaging system may also contain one or more partitions with (different) openings for different tube sizes. For example, a partition may have 1, 2, 3, 4, 5 or more openings. In some examples, a partition has (exactly) one or two openings. If a partition has openings for multiple tubes, all openings of the partition can be configured for the same or different tube sizes (tube circumferences). Common example tube sizes in the medical field are ¼″ or ⅜″, for which the partition may have corresponding openings (¼″ or ⅜″ openings). To avoid confusion of the partitions for different tube sizes, the partition may have a marking corresponding to the tube size. In this way, for example, the tube size can be applied to the partition, e.g. by means of injection molding, so that it is visible when the partition is closed, for example.

For the (friction-locked) insertion of tubes into a partition, it is advantageous, if the partition can be opened (unfolded), e.g. in two (longitudinal) parts (e.g. (longitudinal) halves) of the partition. The axis for opening/unfolding the partition can run (essentially centrally) through the one or more openings of the partition provided for the tubes. This way the tube can be easily inserted when the partition is open/unfolded (into a part of the partition or into a part of the (opened) opening of the partition) and the partition can be closed (with inserted tube). A force-fit or form-fitting lock or closure between the inserted tube and the opening in the partition may only be created when the partition is closed (folded together/closed). For example, the partition may have a joint or hinge. This allows the two (longitudinal) parts ((longitudinal) halves) of the partition to be opened/unfolded and closed/folded.

In this way, the tubes can easily be inserted into an open partition and the partitions can then be closed/folded. A partition can have a labyrinth closure which is particularly advantageous. This closure can have several crossbars. A labyrinth closure thus enables a particularly tight closure of the partition.

The partition can be configured to be gas permeable (in the area outside the openings). In this way, sterilization by means of gas, e.g. ethylene oxide gas, in the packaging can be enabled or facilitated. On the one hand, a particularly secure fixing of the tube in its position can be guaranteed without, on the other hand, influencing the gas supply and removal.

The (closed) partition with the tube(s) inserted in it can then be inserted (e.g., in a form-fitting manner) into a lateral cut-out of a recess in the inlay. This can be performed e.g. by clicking the partition into the inlay.

Furthermore, the packaging system may include fixation means with which components of the tubing system can be fixed in the packaging system (e.g. on/in/at the inlay or a cover). A partition as described above can be used for this purpose. Other non-restrictive examples of fixation means are one or more straps and one (or more) fixing clip(s).

The packaging system can advantageously include one or more (e.g. two) straps. The strap can be used to fix components of the tubing set (or other items to be packaged) on the inlay and/or on one (or more) cover(s). It can also be used to fix a cover on a recess or on a receiving section (with tubing set components).

The strap or straps can be elastic. Various suitable elastic materials are known to the skilled person, e.g. silicone or rubber. Silicones include synthetic polymers in which silicon atoms are linked via oxygen atoms (poly(organo)siloxanes). Rubbers include vulcanizates of natural and synthetic rubbers. Silicone straps can be used in some embodiments. An elastic strap is versatile and allows easy and safe packaging of the tubing set. Thus, objects (e.g. oxygenators) of different sizes can be fixed with the same strap. In this way, the same straps can be used in packaging systems for different tubing sets, i.e. the straps do not have to be adapted separately for each packaging system. An elastic material can also provide additional safety when transporting and storing sensitive objects (such as oxygenators), as the sensitive object (e.g. an oxygenator) is cushioned and held securely in the inlay, thus preventing abrasive wear and scratches caused by vibrations. In addition, an elastic strap allows easy locking and unlocking.

The strap or straps can be fixed on one side (i.e. at one end of the strap) at or in the inlay. On the other side (at the other end), the strap can be fixed to the inlay (e.g. with a defined tightening torque) in such a way that it can be easily released and reattached or refixed, if necessary. For example, the other side (the other end) of the strap can form a (reversible) closure on the inlay, which can e.g. be easily opened and closed.

The strap has two end regions (one at each end of the strap), where an “end region” is the (respective) terminal (end) region of the strap, which is about one third (e.g., about one quarter or about one fifth) of the total length of the strap. Thus, for example, the two end regions of the strap include the two terminal thirds (e.g., the two terminal quarters, the two terminal fifths) of the strap (relative to the total length of the strap).

The strap may have one or more openings in one end region and in the other end region a region (on both sides) of reduced width, followed by a widened region towards the (strap) end (which usually forms the end of the strap). This allows easy fastening (without any additional components) and quick unlocking of the strap (or objects fixed with it). The widened region (at one end of the strap) is wider than the adjacent region (on both sides) of reduced width, e.g. at least 1.5 times or twice as wide. Furthermore, the widened region (at one end of the strap) is wider than the one or more openings in the other end region of the strap, e.g. at least 1.5 times or twice as wide. The region (on both sides) of reduced width can, for example, have a width that is less than or equal to the width of the one or more openings in the other end region of the strap.

The one or more openings can have a smaller width than length (where the terms “width” and “length” of the openings are used analogously to the strap, i.e. the “length” of the openings is measured in the same direction as the length of the strap (longitudinal direction of the strap) and the “width” of the openings in the same direction as the width of the strap). For example, the openings can be at least 1.5 times or twice as long as wide. Hence, the openings can oblong. Multiple openings can be arranged one behind the other (in the longitudinal direction of the strap). Multiple distances between multiple openings can be approximately identical. Multiple openings have the advantage of allowing greater flexibility in terms of fastening objects of different sizes. For example, the strap can have 1, 2, 3, 4, 5, 6, 7, 8, 9, 10 or more openings. In particular embodiments, the openings are not limited to the end region of the strap. For example, the openings can be arranged along the entire length of the strap (e.g. except for the region (on both sides) of reduced width and, optionally, the following end).

To close (fasten) the strap, the end region with the widened region can be passed through one of the openings in the other end region, e.g. until the strap widens again following the region (both sides) of reduced width (so that the region (both sides) of reduced width essentially remains in the opening). The passing through can be enabled, for example, by turning the widened region, which is located adjacent to the region (both sides) of reduced width, e.g. by approx. 90°. After passing through, the widened region adjacent to the region (on both sides) of reduced width can be turned back to its original position (or, e.g. in case of an elastic strap, turns back to its original position itself). The widened region at one end of the strap prevents an unintentional or independent opening of the strap (slipping of the strap end out of the opening through which it was guided).

For reversible opening of the strap, the widened region can be passed through one of the openings in the other end region (e.g. by turning it, e.g. by approx. 90°), wherein the strap is passed through in the opposite direction as compared to the passing of the strap when closing it.

To facilitate the passage of the widened region, its side edges may have a rounded or beveled or tapered shape. For example, the widened region of the strap can have a substantially hexagonal shape.

The inlay (or cover) can have one or more openings (e.g., (exactly) two per strap) through which the strap can be passed. These openings in the inlay are used to fix objects in or on (the recess/receiving section) of the inlay or cover with the strap. The openings in the inlay (or cover) can be at least as long as the strap is wide and/or at least as wide as the strap is thick.

In order to prevent or minimize horizontal slippage of the strap (e.g. during transport or storage), a cover may include a guide for the strap. Thus, the packaging system may have a cover for a recess/receiving section in the inlay and/or for a recess/receiving section in another cover that has a guide for a strap. In this way, the strap can be secured against unintentional horizontal movement. Such a cover can e.g. be arranged (essentially form-fitting) on the inlay or on a (further) cover (e.g. a cover with recesses for receiving additional objects or items that can be used in connection with the medical tubing set).

The strap can also be used to suspend the inlay advantageously. For this purpose, the (elastic) strap can be anchored on one side (tension) firmly in the inlay. Similarly, e.g. objects such as the oxygenator can be attached to the inlay by means of a strap. After removing the objects, the inlay can be suspended on the (elastic) strap—e.g. on a holder of the console (the control unit for extracorporeal therapy) or on an infusion hook. Especially when the inlay is suspended, at least a part of the (blood-carrying) tube, if required, can remain in the inlay during use (e.g. during preparation and/or execution of the extracorporeal therapy). Alternatively, the (blood-carrying) tube 20 can also be removed from the inlay for use.

Furthermore, the packaging system can advantageously include a fixing clip. The fixing clip can be configured in such a way that it at least partially encloses a component of the tubing set (or part of such a component) (e.g. by non-positive or positive locking). For example, the fixing clip for use on components with essentially round cross sections/shapes may have a corresponding rounding which at least partially encloses the essentially round shape/cross section of the object to be fixed. For example, the rounding of the fixing clip can enclose more than half (e.g. about two thirds) of the essentially round shape/section of the object to be fixed. Thus, the object to be fixed can be fixed in the fixing clip. In addition, the object to be fixed can simply be guided into the fixing clip (and received by it). The clip can advantageously also be configured for different tube diameters (e.g. for two different tube diameters). The fixing clip can also have extensions in the lower part, by means of which it can be fixed in corresponding openings in the inlay (or in a cover). Accordingly, the inlay (or a cover) may have openings for fixing a fixing clip. The fixing clip can e.g. be fixed in the inlay by pressing the clip together and may subsequently be removed again. The fixing clip can be made of plastic, e.g. polypropylene.

The packaging system can be advantageous in that it allows unpacking essentially in the order in which the packaged items are used. In other words, the tubing set can be arranged in the packaging system in a way that allows unpacking essentially in the order of use (e.g. in the order in which the tubing set is assembled or provided for extracorporeal therapy). In particular, the packaging system can enable packaging in which the user only sees those items or objects that are needed in the next step of use. In this way, mistakes during assembly and/or use of the packaged objects can be avoided (Poka-Yoke principle).

For this purpose, the components of the tubing set can be advantageously packaged in such a way that those components which are used first are packaged last (e.g. if possible on top) and can be unpacked first (e.g. by placing them at a top position in the packaging). On the other hand, components of the tubing set that are later (or ultimately) used can be packed further down (or at the very bottom). For example, different compartments can be created by one or more covers in the packaging system, wherein the packaged items packed on (above) a cover are visible first and are therefore usually removed from the packaging system first when unpacking. On the other hand, objects packed under a cover are intuitively unpacked later (after removing the cover and the objects arranged thereon). This type of packaging allows the user to intuitively remove the packaged tubing set step by step in the order of use.

For example, the inlay may have recesses to accommodate a blood-carrying tube (i.e. a tube for blood conduction). Due to the sterilization process, such tubes are usually delivered open (at their ends) and must be connected accordingly at the place of use, e.g. to an oxygenator. Therefore, it is advantageous to protect tubes in which the patient's blood is to be conducted, even in sterile areas, especially from contamination such as the smallest dust particles, since the blood (and thus possible contamination) guided therein is returned to the patient. In order to avoid long periods of time between the removal of blood-carrying tubing from the packaging system and its use (and thus an increasing probability of contamination), it is advantageous to unpack blood-carrying tubing at the very end (or as one of the last tubing set components) when unpacking the tubing set, so that it can be directly connected (and prepared for extracorporeal therapy). Therefore, it is advantageous, if the inlay has corresponding recesses to accommodate a blood-carrying tube, such that thereby the “lowest” or at least one of the lower levels of packaging is established.

In a further aspect, the present disclosure also provides the use of the packaging system described herein for packaging a medical tubing set. As described above, the packaging system described herein is configured for this purpose, such that detailed and features of the packaging system are also applicable to its use for packaging a medical tubing set.

In a further aspect, the present disclosure provides a method for packaging a medical tubing set in a packaging system as described herein, said method including the following steps:
(i) packaging the medical tubing set on the inlay outside the tray;
(ii) inserting the inlay with the packaged tubing set into the tray; and
(iii) reversible fastening of the inlay in the tray.

The detailed description of the packaging system described herein is also applicable to the method described herein. In particular, the packaging system described herein enables a medical tubing set to be packaged using the method described herein, wherein in particular the tubing set can be easily packaged outside the tray on the inlay and may subsequently be inserted into the tray and be reversibly fastened.

Thus, the method described herein may include, for example, the attachment or fastening of a fastening means to the inlay. Thus, in the case of a multi-part fastening means (e.g. with an upper and a lower part), one of the parts (e.g. the upper part) can be passed through an opening provided for this purpose in the inlay and then connected to the other part (e.g. the lower part) (e.g., by snapping in or by connecting an extension of one part of the fastening means to a receptacle provided for this purpose in the other part of the fastening means).

Furthermore, the method can include equipping covers, if provided, e.g. with the respective components intended for this purpose (e.g. additional items that are usually used together with the medical tubing set, such as tubing clamps, a syringe, cable ties, etc.).

If the packaging system has one or more straps, they can be initially attached to the inlay in the method described herein by passing them, e.g., through openings in the inlay arranged for this purpose. If necessary, components of the tubing set (e.g. an oxygenator) that are to be attached, e.g., by means of a strap directly to the inlay can then be attached with the strap. For this purpose, the component (e.g. an oxygenator) can be inserted into the recess or the receiving section of the inlay provided for this purpose and can then be secured with the strap.

In the method described herein, the recesses (or receiving sections) of the inlay can also be equipped with (the designated) components of the tubing set. For example, a tube can be rolled up in a corresponding recess of the inlay. Tube ends can be guided around other components, such as the oxygenator. Open tube ends can be inserted into a (coverable) recess. Partitions can be used for fixation and—especially with open tube ends—for further protection, as described above. For example, open tube ends that require particular protection can first be inserted into partitions that are inserted into the inlay (in recesses or cut-outs provided for this purpose), so that a particularly protected space for the open tube ends is created by means of partitions and a cover. Other components, such as a pump head, can be aligned on the inlay as required, e.g. with an outer housing essentially parallel to an edge of the inlay. If required, components of the tubing set can be attached to the inlay in various ways, e.g. with a fixing clip, with a strap or with a partition as described above.

After equipping the recesses or receiving sections of the inlay, they can be covered with an appropriate cover, if provided. The covers can, for example, be attached directly to the inlay. It is also possible to arrange multiple covers (which are each equipped as described above) on top of each other. In this case, covers that are arranged above another cover can be attached directly to the inlay or to the lower cover. If necessary, further components can then be arranged on the covers. The cover(s) can also be attached (e.g. after equipping or assembly) by means of a strap, so that the cover is initially only placed on top.

In the described herein, the (completely) equipped inlay is placed in the tray and the equipped inlay is reversibly fixed therein. The fastening of the (equipped) inlay in the tray can be done as described above, e.g. with the fastening means described above. The user can e.g. move the fastening means arranged at the inlay or the upper part of the fastening means, such that a locking mechanism is created in the tray, e.g. by rotating the fastening means (e.g. by means of wings on the fastening means and corresponding projections in the tray).

In particular, the tray can be sealed (e.g., with a foil, as described above) after the inlay has been fastened in the tray. The (sealed) tray can be packed in secondary packaging (e.g. a dust bag and/or an outer carton).

In a further aspect, the present disclosure provides a fastening means for reversibly fastening an inlay in a tray, the fastening means including a handle and a base, the handle, e.g., having a gripping portion and an extension for connection to the base which can be passed through an opening in the inlay. In some embodiments, the handle incorporates a hook (for attachment to a rail or rod) which can be released from the handle.

### BRIEF DESCRIPTION OF THE DRAWINGS

The systems, methods, and devices described herein are explained in more detail below on the basis of the embodiments shown in the figures. The figures and the following description shall illustrate the systems, methods, and devices in an exemplary manner without limiting the subject matter of the disclosure. It is shown in:

FIG. 1 a schematic representation (exploded view) of an embodiment of a packaging system.

FIG. 2 a schematic representation of the packaging system with medical tubing set shown in FIG. 1, wherein the tray and the inlay with the tubing set packed on it are shown separately for better overview.

FIG. 3 a schematic representation of the packaging system with tubing set shown in FIG. 1 in top view, wherein the inlay with the packed tubing set is arranged in the tray.

FIG. 4 a schematic representation of a tray of a packaging system in top view.

FIG. 5 a schematic representation of the front section of a tray of a packaging system in a longitudinal section along the plane A-A shown in FIG. 4.

FIG. 6 a schematic representation of the tray with inlay shown in FIG. 4 and its fastening with a fastening means in the tray.

FIG. 7 a schematic representation of the fastening of an inlay in a tray with a further embodiment of a fastening means (A); a schematic representation of a handle of a further embodiment of a fastening means (B); and a schematic representation of a handle of a further embodiment of a fastening means (C).

FIG. 8 a schematic representation of a further embodiment of a fastening means (A); a schematic representation of the handle of the fastening means (B) shown in (A); and a schematic representation of the base of the fastening means (C) shown in (A).

FIGS. 9-12 respective schematic representations of different embodiments of a partition in closed (A) and open (B) state.

FIG. 13 an exemplary embodiment of a (elastic) strap that can be used to fix objects (components of the tubing set) in the packaging, especially in the inlay.

FIG. 14 an example of a fixing clip in perspective view (A) and top view (8) that can be used to fix objects (components of the tubing set) in the packaging, in particular on the inlay.

FIG. 15 a schematic perspective representation of an exemplary cover for a recess in the inlay of a packaging system.

FIG. 16 a schematic perspective representation of a further exemplary cover of a packaging system.

FIG. 17 a schematic representation of an exemplary inlay of a packaging system in a perspective (A), a top view (B), and the section indicated in FIG. 17B in enlargement (C).

FIG. 18 a schematic representation of a further exemplary tray of a packaging system in a top view (A) and the section A indicated in FIG. 18A in enlargement (B).

### DETAILED DESCRIPTION

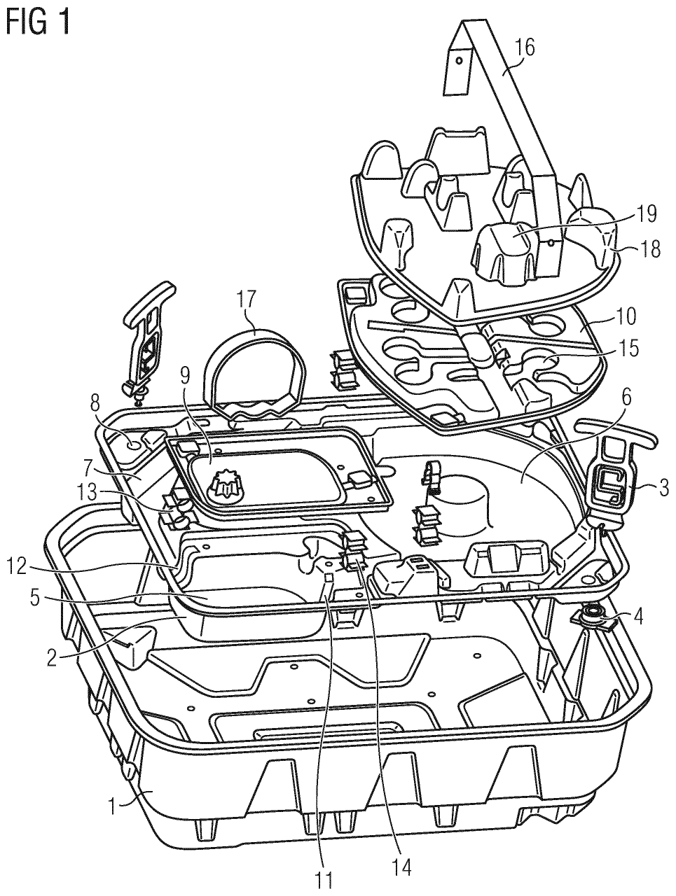

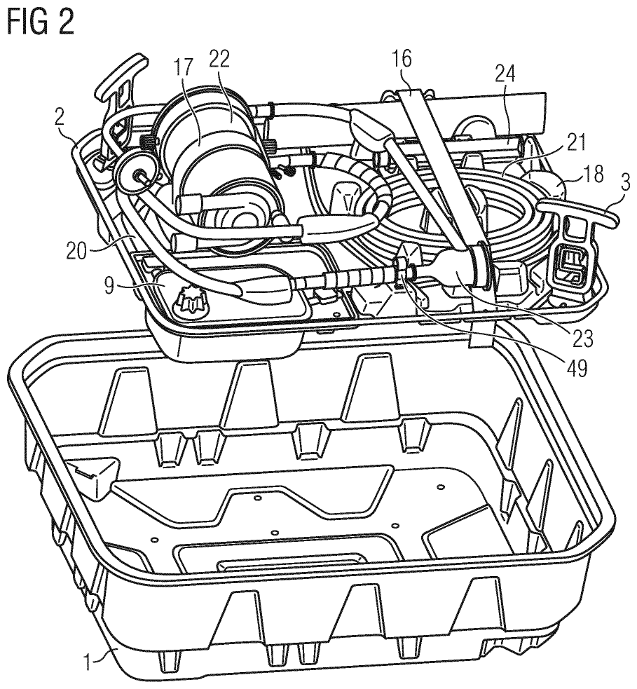

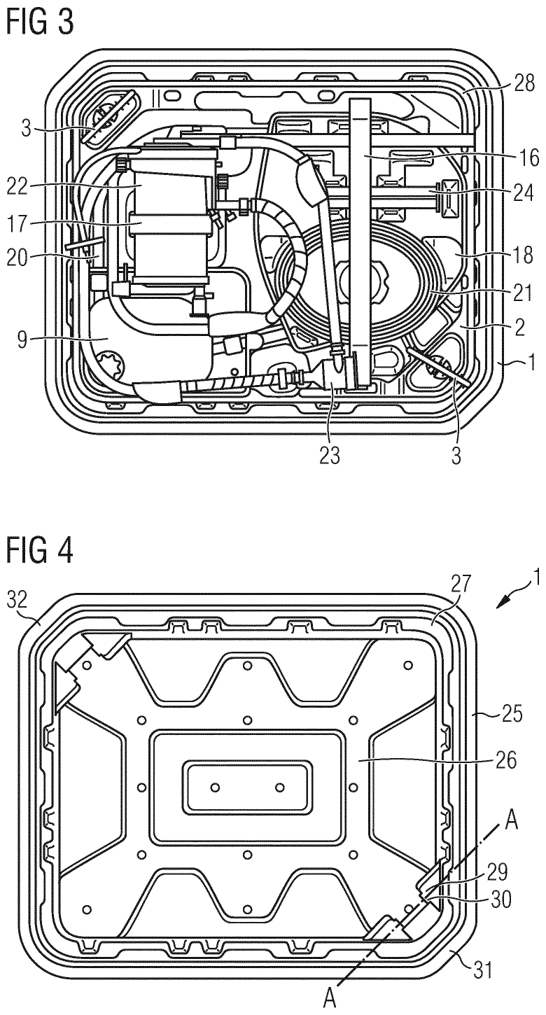

A packaging system for a medical tubing set for extracorporeal therapy with a tray 1, an inlay 2 and a fastening means 3, 4 for reversible fastening of the inlay 2 in the tray 1 is shown in FIGS. 1 to 3. FIG. 1 shows the packaging system without the tubing set; however, FIGS. 2 and 3 each show the packaging system with an exemplary medical tubing set for extracorporeal therapy.

The inlay 2 has recesses 5, 6, 7 to accommodate a tube. Furthermore, the inlay may have a receiving section for an oxygenator (shown in FIGS. 2 and 3 with oxygenator). In addition, the inlay 2 may have an opening 8 through which the fastening means 3, 4 can be passed or guided in order to fix the fastening means 3, 4, e.g., rotatably to the inlay 2. The inlay 2 may advantageously include a respective opening 8 in the diagonally opposing corners 50, 51 of the inlay 2, such that a fastening means 3, 4 may be respectively arranged in the diagonally opposing corners 50, 51 of the inlay 2. A fastening or attachment in (exactly) two diagonally opposing corners 50, 51 of the inlay 2 provides for a secure fixation of the inlay 2 in the tray 1. The inlay 2 can also have openings for a strap 16, 17. In addition, the inlay 2 can have openings for the attachment of a fixing clip 49.

In addition to the tray 1, inlay 2 and fastening means 3, 4, the packaging system may have one or more covers 9, 10 for recesses 5, 6 in the inlay 2. One or more recesses 5, 6 in the inlay 2 may not be completely closed laterally (in covered state), but may have a lateral cut-out or recess 11,12 at least on one side of the recess 5, 6, so that one or more tubes can be inserted or removed laterally into or from the recess 5, 6. In this way, two or more recesses 5, 6, 7 can be connected to each other so that one tube can be led through several recesses 5, 6. In other words, different interconnected recesses 5, 6 can be used to receive the same tube. Furthermore, the packaging system can include one or more partitions 13, 14, which on the one hand can fill the lateral recesses 11, 12 of the recess 5 in a form fitting manner and which on the other hand can have openings for the possibly accurate passage of tubes. In this way, such partitions 13, 14 can be used to form a, e.g., closed, interior space in a recess 5 of the inlay 2 with the cover 9, which is particularly protected against contamination and into and out of which one or more tubes can nevertheless be guided (essentially non-positively or force-fit) through the openings of the partitions 13, 14. Furthermore, the partitions 13, 14 can be used to lock the tubes in the packaging system. Furthermore, the packaging system can have a cover 10 for a recess 6 in the inlay 2, which by itself has recesses 15, which, for example, serve to accommodate (further) elements of the tubing system (e.g. additional tubes) or to accommodate additional items, such as tube clamps.

In order to fix the tubing set in the inlay 2, the packaging system can include one or more (e.g. two) straps 16, 17, which are, e.g., elastic (e.g., made of silicone). Multiple straps 16, 17 can differ, for example, in length, width, shape and/or material or have the same length, width, shape and/or material. For example, multiple straps 16, 17 may differ only in color (and otherwise be identical in construction). The strap or the (elastic) straps 16, 17 can be anchored and (tensionedly) held on one side together with or in the inlay 2. On the other side, the (elastic) straps 16,17 can be attached to the inlay 2 in such a way that they can be easily detached and fastened if required. To prevent or minimize horizontal slipping of such a strap (e.g. during transport or storage), a guide 19 for the strap 16 can be provided in a cover 18.

The exemplary tubing set sterilely packed in the packaging system includes a tube for blood conduction 20, a tube for gas conduction 21, an oxygenator 22, as well as a pump head 23. Furthermore, various additional items that are usually used together with the medical tubing set, such as tube clamps, a syringe 24, cable ties, etc., can also be packed (in a sterile manner) in the packaging system.

As shown in FIG. 2, the tubing set can be packed completely on inlay 2, especially outside of the tray 1. When the tubing set is packed on inlay 2 outside of the tray 1, the elements of the tubing set and potential additional items are easily accessible and can therefore be easily packed on inlay 2. Afterwards, inlay 2 (with the tubing set packed on it) can be placed into tray 1 and reversibly fastened therein with fastening means 3, 4 as shown in FIG. 3. In this way, not only a particularly easy packing but also a particularly easy unpacking is enabled, since the reversible fastening 3,4 of the inlay 2 in the tray 1 allows the inlay 2 with tubing set components to be easily removed from the tray 1 and the tubing set components to be brought to their destination outside of the tray 1 and be unpacked. Simple packaging and easy unpacking with easy access to the components is particularly advantageous under sterile conditions, such as packaging in a clean room and/or unpacking in an intensive care unit. After the tubing set has been packed in the packaging system, the packaging system can be sealed (sterile), e.g. by applying a cover, e.g. a foil, to the tray 1 for (sterile) sealing (of the contents). For transport, the sealed tray 1 can be packed in a secondary packaging, e.g. in an outer box.

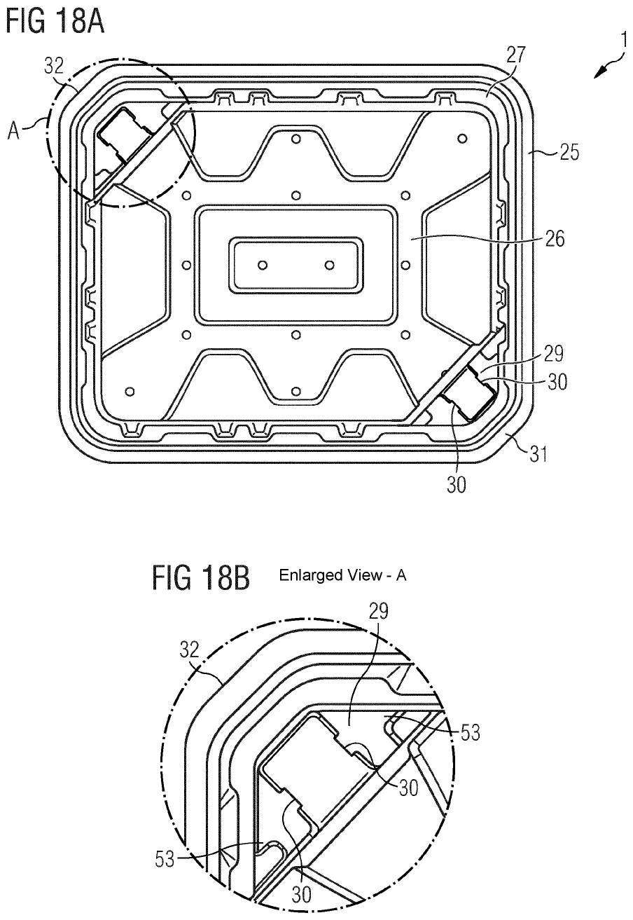

As shown in FIGS. 4 and 18, the tray 1 can advantageously have an essentially horizontal upper edge 25, such that the cover (foil) can be applied easily. Such an edge can, for example, have a width of approx. 5 to 20 mm.

As shown as an example in FIGS. 4 and 18, the tray 1 can have an essentially rectangular base surface (bottom 26), wherein the “corners” of the rectangle can be rounded or beveled/tapered. The walls of the tray 1 are essentially perpendicular to bottom 26 of the tray 1. As shown in FIGS. 1-4 as an example, the wall of tray 1 can have a gradation or step so that the base area (at bottom 26) of tray 1 is smaller than the (inner) area formed by the upper edge 25. Such trays can be easily stacked and therefore stored in a space-saving manner.

The tray shown in FIG. 4 has an essentially horizontal support surface 27 for the inlay 2 in the lower half of the wall height (e.g. at approx. one third of the wall height). In FIG. 4, this support surface 27 is essentially circumferential. The support surface 27 can be achieved, for example, by a step in the (inner) wall. When packing the tubing set, the inlay 2 is placed on the supporting surface 27 of the inner wall of the tray 1 so that the underside of the outer edge 28 of the inlay 2 is at least partially in contact with the supporting surface 27 of the tray 1. The dimensions of the tray 1 are such that the inlay 2 can rest simultaneously on the contact surfaces 27 on all inner wall sides (e.g. on all four inner wall sides in case of a rectangular basic shape in FIG. 4) and the inner walls of the tray 1 thus limit or essentially prevent a horizontal movement of the inlay.

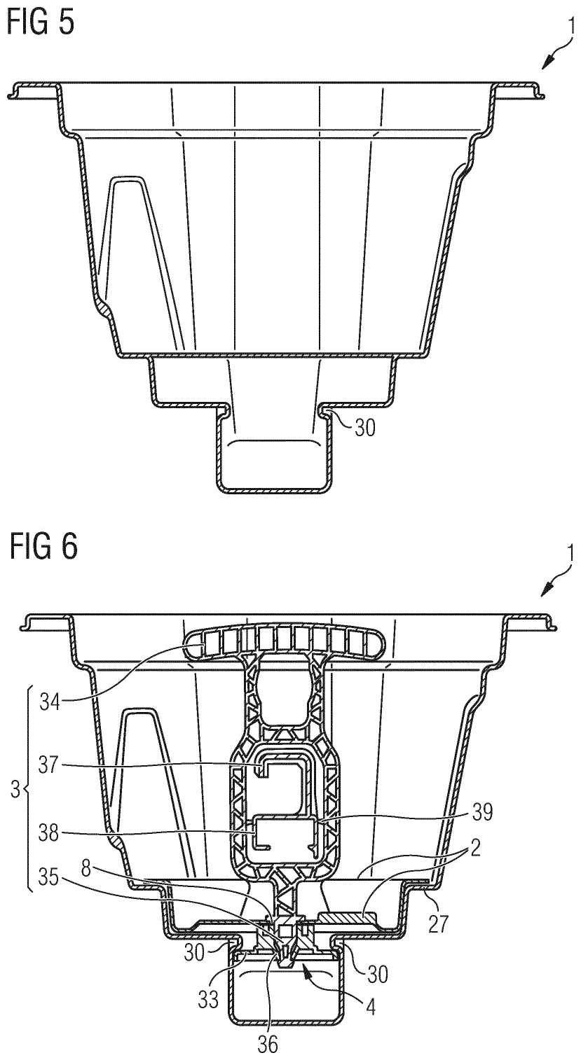

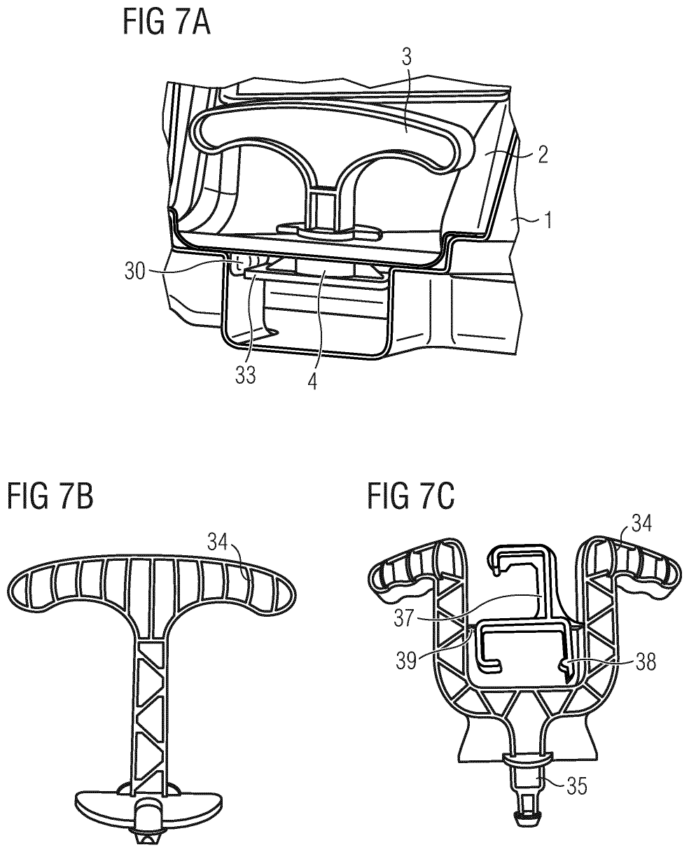

As shown in FIGS. 4 and 18 as an example, the tray 1 can advantageously have a receptacle 29 in the region close to the bottom or floor for locking by means of fastening elements (fastening means) 3, 4. This receptacle 29 is formed by a correspondingly dimensioned projection 30 in the (inner) wall of tray 1, as shown in FIGS. 5 and 6 in longitudinal section (through a corner of the tray). As shown in FIGS. 4 and 18, two receptacles 29 with (e.g. two) projections 30 can be arranged in the diagonally opposite corners 31, 32 of tray 1 if tray 1 is rectangular. Exactly two receptacles 29 can be provided to fix the inlay 2 in the tray 1. In the exemplary tray 1 shown in FIGS. 4, 5 and 6, the receptacle 29 for fixing the inlay 2 in the tray 1 has two, in particular opposite, projections 30 which are dimensioned in such a way that the wings 33 of a base 4 of the fastening means 3, 4 arranged essentially below the inlay 2 engage at least partially beneath the projections 30, when the fastening means 3, 4 is moved (e.g. rotated). This is illustrated in FIGS. 6 and 7a as an example. When the fastening means 3, 4 is moved in the opposite direction (e.g. rotated in the opposite direction), the wings 33 of the base 4 of the fastening means 3,4 no longer reach beneath the projections 30. The interaction of the projections 30 of the (inner) wall of the tray with the wings 33 of the base 4 of the fastening means 3, 4 therefore enables locking by moving the wings 33 relative to the projections 30. When turning the fastening means 3, 4 for reversible fastening of the inlay 2 in the tray 1, for example, a rotation of about 90′ can be performed to fasten or release the inlay in the tray.

FIGS. 6 and 7a show that the fastening means 3, 4 for fastening the inlay 2 in the tray 1 is usually connected to the inlay 2, wherein the fastening means in the inlay 2 can be movable, e.g. rotatable.

The connection or attachment of the fastening means 3, 4 with/to the inlay 2 can be reversible or irreversible.

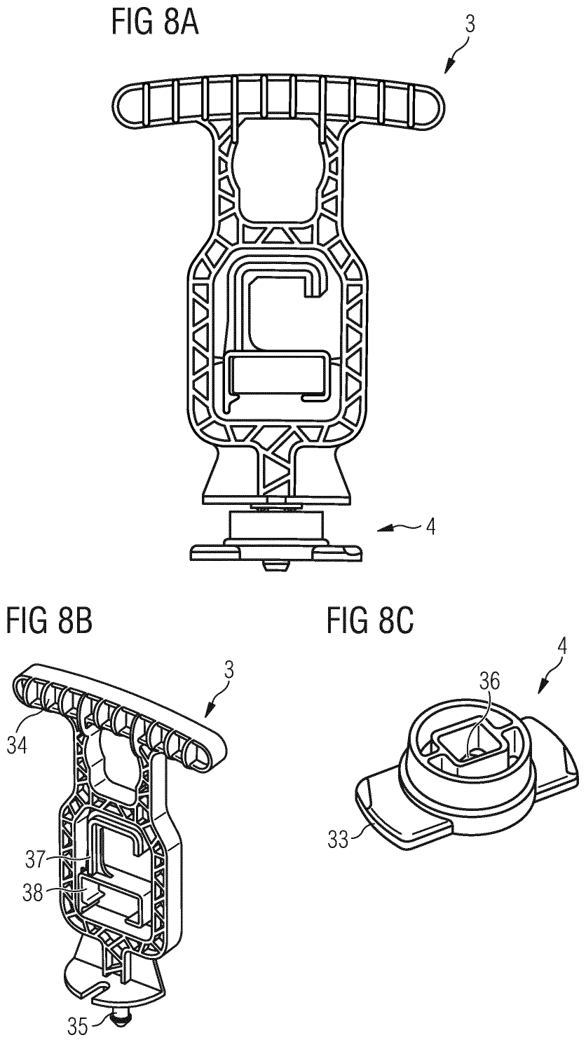

It is advantageous if the fastening means has a handle 3 and a base 4 as exemplary shown in FIGS. 1, 6, 7a, and 8a. The base 4 of the fastening means can be arranged essentially below the inlay 2 (or additionally also within the inlay 2). The base 4 of the fastening means can, for example, be used for locking purposes, e.g. in combination with the projections 30 of the tray wall, as described above. For this purpose, the base 4 of the fastening means can have one or two wings 33, as shown in FIG. 8c by means of example. Such a wing 33 can be positioned beneath the projections 30 of the (inner) wall of tray 1 when the fastening means is rotated. This can essentially prevent vertical movement of the inlay 2 out of the tray 1.

The handle 3 of the fastening means can be positioned essentially above (or additionally also in) the inlay 2. The handle 3 of the fastening means can, for example, be used by the user to operate the fastening means, e.g. to fasten or release the inlay 2 in the tray 1 (e.g. by manually operating the fastening means).

A small sub-portion of the base 4 of the fastening means can also be arranged above the inlay 2 and/or a small sub-portion of the handle 3 of the fastening means can also be arranged below the inlay 2, e.g. to connect both parts 3, 4 of the fastening means, as shown in FIGS. 6, 7c, and 8b. Here, the handle 3 of the fastening means 3, 4 has an extension 35 next to the gripping section 34, which (when the fastening means 3, 4 is fastened to the inlay 2) extends through the opening and below the inlay 2 to connect the handle 3 to the base 4. In other words, the extension 35 of the handle 3 of the fastening means can be passed through (or into) an opening 8 of the inlay 2 to connect the handle 3 of the fastening means on the top of the inlay 2 to base 4 of the fastening means on the bottom of the inlay 2, as shown in FIG. 6 as an example. For this purpose, base 4 of the fastening means advantageously includes a seat 36 or receptacle for the extension 35 of the handle 3 of the fastening means, as shown in FIGS. 6 and 8C. By inserting the extension 35 into the seat 36, a suitable connection of the handle 3 and the base 4 of the fastening means is achieved. Such a connection will generally allow movements, such as a rotary movement, of the fastening means handle 3, in particular the gripping section 34 of the fastening means, to be transferred to the fastening means base 4. If, for example, the user performs a rotational movement on gripping section 34, this will also rotate base 4 of the fastening means (arranged below the inlay 2). In this way, the user can rotate the wings 33 of the base 4 of the fastening means, e.g. by turning the gripping section 34, so that these wings 33 are positioned beneath the projections 30 of the (inner) wall (and thus the inlay 2 is fixed in the tray 1). Similarly, a (further or opposite) rotation of the gripping section 34 can release the fastening of the inlay 2 in the tray 1 if the wings 33 of the base 4 of the fastening means are no longer positioned beneath the projections 30 of the (inner) wall. Such a locking mechanism can be configured in such a way that it engages in a position that is as optimal as possible (e.g. with the greatest possible overlap of wings 33 and projections 30; e.g. after turning by approx. 90′). As shown in FIG. 8c, the outer side edges of the wings 33 may have a rounded or chamfered/beveled/tapered shape.

FIGS. 6-8 show various exemplary shapes of the gripping section 34, wherein these exemplary gripping sections are sufficiently wide that they can be easily operated with one hand (e.g. at least 3, 4 or 5 cm). The handle 3 with gripping section 34 shown in FIG. 6 is advantageously dimensioned so that the gripping section 34 is arranged in an upper section of the tray 1, i.e. just below the tray edge 25 (so that the upper side of the handle 3 of the fastening means just does not project beyond the upper tray edge 25). This way the gripping section 34 can be easily accessed and is easy to operate. FIG. 7a shows an alternative gripping section 34, which is provided in a middle or lower section of the tray 1.

As shown in FIGS. 6, 7c and 8 as an example, the fastening means 3, 4, especially its handle 3, may contain a hook 37 which can be released (e.g. broken out) from the fastening means 3, 4 if required. The hook 37 may have a holder 38 for fastening the hook 37 to a rod or rail, for example, as shown in FIGS. 6, 7c and 8. In the embodiments shown in FIGS. 6 and 7c, the hook 37 is integrated in the fastening means 3, 4 (or in its handle 3) only via a few (e.g. two or three) connecting webs or bars 39 which are as thin as possible. Thus, neither fastening means 3, 4 nor hook 37 will be damaged when hook 37 is removed from fastening means 3, 4. As shown in FIGS. 6, 7 and 8b, the handle 3 of the fastening means 3,4 with the integrated hook 37 can advantageously be made in one piece so that the handle 3 with the integrated hook 37 can be produced in the same process step. In case of a one-piece design, the hook 37 is usually made of the same material as handle 3.

When fixing the inlay 2 in the tray 1 with the fixing material 3, 4, a cavity is formed between tray 1 and inlay 2. This cavity serves for the safe transport of sensitive objects (e.g. the oxygenator 22 and/or the pump head 23), since the cavity between inlay 2 and tray 1 can absorb vibrations to protect the tubing set.

It is advantageous to arrange the tubing set on inlay 2 in such a way that the tubing set can be unpacked essentially in the order of use (e.g. in the order in which the tubing set is set up or made available for extracorporeal therapy). In this way, errors in the use of the tubing set can be avoided (Poka-Yoke principle). For this purpose, the components of the tubing set that are used first can be packed at the top, if possible, while components of the tubing set that are used later (or last) can be packed further down (or at the bottom).

In some embodiments, inlay 2 can also be advantageously suspended—for example, by means of a (elastic) strap 16, 17 of the packaging system. For this purpose, the (elastic) strap 16, 17 can be anchored on one side and firmly (e.g., firmly via tension) held in the inlay 2 (for example, objects such as the oxygenator 22 can also be attached to the inlay 2 with strap 16, 17). Especially when the inlay 2 is suspended, at least part of the (blood-carrying) tube 20 can remain in the inlay 2 during use (e.g. during preparation and/or performance of extracorporeal therapy), especially in the recesses 5, 6, 7 of the inlay 2, which are formed/configured for the (blood-carrying) tube 20.

The recesses 5, 6, 7 in the inlay 2 (or in one or more covers 9, 10, 18) provided for tubes can advantageously be dimensioned larger than the tube circumference. The recesses 5, 6, 7 in the inlay 2 (or in one or more covers 9, 10, 18) may be dimensioned to accommodate multiple layers of the tube or multiple tubes. It is advantageous, for example, to have essentially round or elliptical recesses or receptacles for tubes, wherein a raised area or elevation can be arranged approximately centrally to wrap the tube around it. This is shown in FIG. 1 for the recess 6 in the inlay 2 as well as for the cover 18, on whose elevation the guide 19 for strap 16 is arranged.

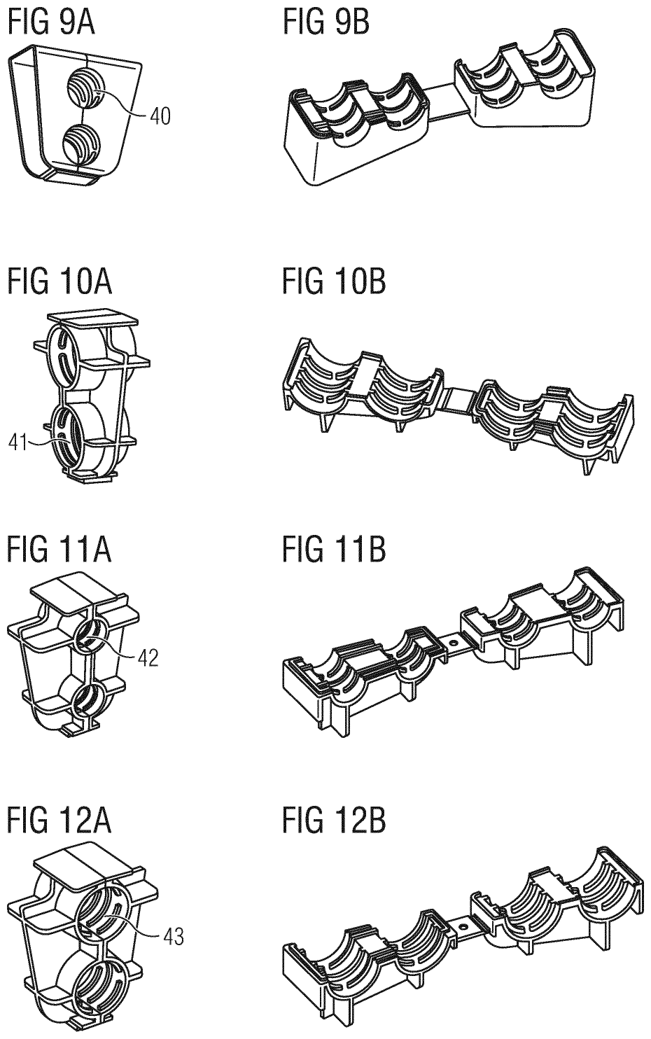

One or more recesses 5, 6 in inlay 2 may be provided to form particularly protected compartments in the packaging system by means of a cover 9, 10 and suitable sealing means 13, 14. In order to guide a tube into or out of such a particularly protected compartment, one or more partitions 13, 14 may be arranged in lateral recesses or cut-outs 11, 12 of the recess 5, 6 (see FIG. 1). The partitions 13, 14 can fill or fit the lateral cut-outs 11, 12 of the recesses 5, 6 in a form-fitting manner on the one hand and have openings 40, 41, 42, 43 on the other hand to allow tubes to pass through them as accurately as possible. Exemplary partitions are shown in FIGS. 9 to 12. Usually exactly one tube 20, 21 is guided through exactly one opening 40, 41, 42, 43 of a partition 13, 14. The opening 40, 41, 42, 43 of the partition 13,14 is exactly adapted to the tube circumference (tube size) to avoid contamination of the interior of the recess. Thus, a packaging system may have one or more partitions 13, 14 with all openings 40, 41, 42, 43 for the same tubing size, e.g. a (blood-carrying) tubing 20. Alternatively, a packaging system may also contain one or more partitions 13, 14, which have openings 40, 41, 42, 43 for different tube sizes. FIGS. 9 to 12 show advantageous partitions with openings 40, 41, 42, 43 for two tubes each. However, partitions 13,14 for less (i.e. only one tube) or more tubes (e.g. for three or four tubes) are also conceivable and can be realized in a similar way. If a partition 13,14 has openings 40, 41, 42, 43 for multiple tubes, all openings 40, 41, 42, 43 of the partition can be designed for the same or for different tube sizes (tube circumferences).

FIGS. 9-12b show as an example that the partition 13, 14 can be opened advantageously along (through) the openings 40,41,42,43, e.g. in two (longitudinal) halves of the partition 13,14 to facilitate the insertion of the tubes into the partition 13, 14. In this way, the tubes can be easily inserted into the open partitions 13, 14 and the partitions 13, 14 can then be closed. The closed partition 13, 14 with the tube(s) inserted therein can then be inserted (form-fitting) into the cut-outs 11,12 of the inlay 2, e.g. by clicking the partition 13,14 into the inlay 2.

The partitions 13, 14 can be used (e.g. in combination with a cover 9) to form an, e.g., closed, interior space which is particularly protected against contamination and into which one or more tubes can still be guided (in an essentially non-positive manner) through openings 40,41, 42, 43 of the partitions 13, 14.

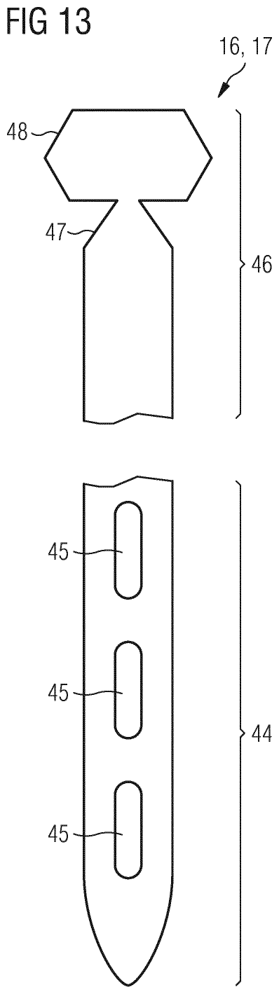

FIG. 13 shows an exemplary embodiment of the strap 16, 17, which can be used to fix objects (components of the tubing set) e.g. in the inlay 2, as described above. Such a fixing of components of the tubing set with a strap 16, 17 is shown as an example in FIGS. 2 and 3. For easy attachment (without any additional components) and for quick release, the strap 16, 17 may have openings 45 in one end region 44 and a region (on both sides) of reduced width 47 in the other end region 46, which is closed off by a widened region 48 towards the (strap) end.

If the strap 16, 17 is formed in this way, the end region 46 with the widened region 48 can be passed through one of the openings 45 in the other end region 44 to close (fasten) it, e.g. until further passage is not possible or difficult, since the strap widens again after the region (on both sides) of reduced width 47. Afterwards, the widened region 48 at one end of the strap prevents unintentional opening (slipping of the strap end out of the opening through which it was passed) of the strap 16, 17. To facilitate the passage of the widened region 48, its side edges may have a rounded or beveled or tapered shape. For example, the widened region 48 may have a substantially hexagonal shape as shown in FIG. 13.

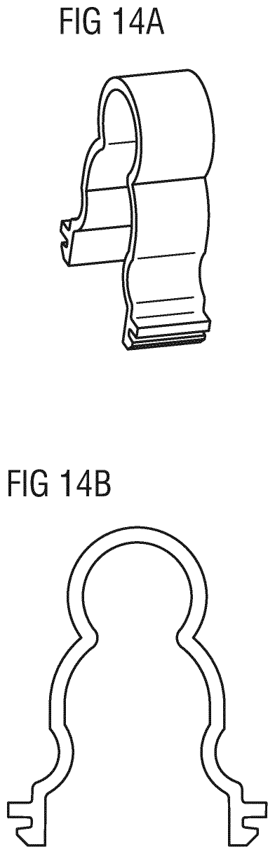

In addition, or as an alternative to fixing with a strap 16, 17, a fixing clip 49 can also be used to fix objects (components of the tubing set) in the packaging. Such a fixing clip 49 is shown as an example in FIG. 14. For example, the fixing clip 49 can be used to fix the pump head 23 on the inlay 2 as shown in FIG. 2. In this way, the fixing clip 49 prevents the pump 23 from moving during transport which could damage other objects or damage the packaging. By pressing the clip 49 together, the clip 49 can be fixed in the inlay 2 and may then be removed again. The clip 49 can be advantageously configured for different tube diameters (e.g. for two different tube diameters as shown in FIG. 14).

FIG. 15 shows an exemplary cover 10 for a recess 6 in the inlay 2. The cover 10 may serve to cover a recess 6 of the inlay 2 (e.g. a tube being supported therein). Furthermore, the cover 10 by itself may include one or more recesses for receiving or arrangement of components of the tubing set or additional items, which may be used in accordance with the medical tubing set, in an essentially form-fitting manner. Accordingly, the exemplary cover depicted in FIG. 15 includes recesses 15 for receiving tube clamps.

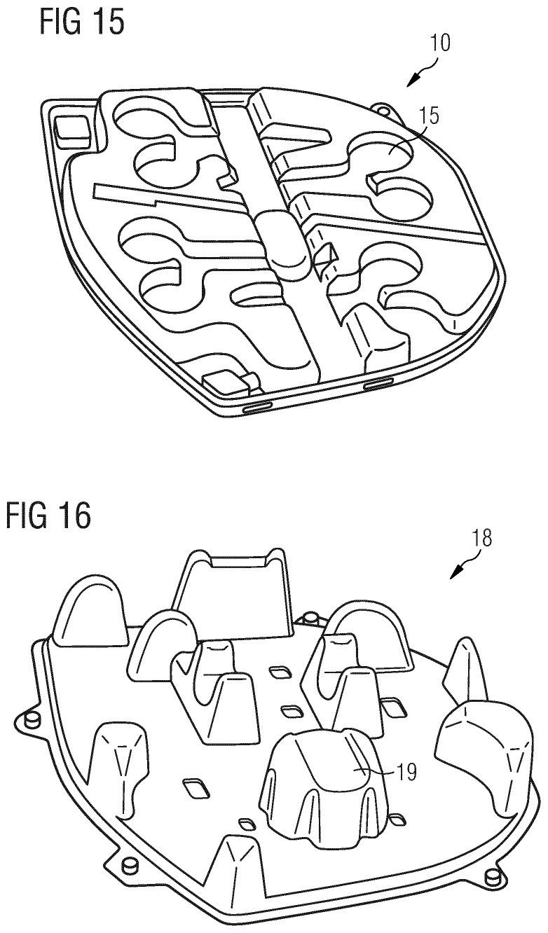

FIG. 16 shows a further exemplary cover 18, which may be adapted as a cover for the cover 10, as shown in FIG. 1. In this manner multiple covers may be arranged (stacked) on top of each other in a space-saving manner, wherein components of the tubing set or additional items may be advantageously separated from each other and packaged in a clearly presented manner. The cover 18 arranged at the top may include a guide 18 for the strap 16. Accordingly, the cover 18—as well as any potential cover(s) being arranged underneath—may be fixed by means of the strap 16. Also the cover 18 may by itself include one or more recesses or elevations or protrusions for receiving or arranging components of the tubing set or additional items, which may be used in accordance with the medical tubing set, in an essentially form-fitting manner. Accordingly, the exemplary cover depicted in FIG. 16 includes elevations or protrusions for receiving/arranging a syringe 24. Furthermore, the guide 19 may be arranged on an essentially circular or elliptical elevation or protrusion, which by itself enables the accommodation or arrangement of a (coiled) tube 21 (around the elevation). The recesses or elevations or protrusions for receiving components of the tubing set or additional items are advantageously arranged in such manner that the items being arranged therein may also be additionally fixed by the strap that is guided through the guide 19.

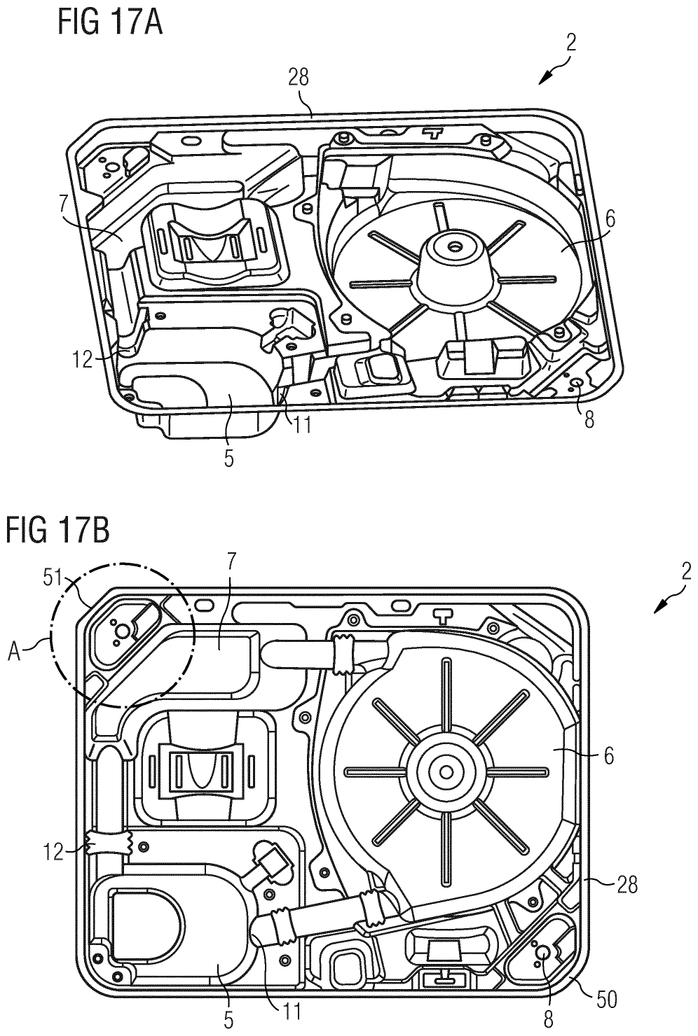

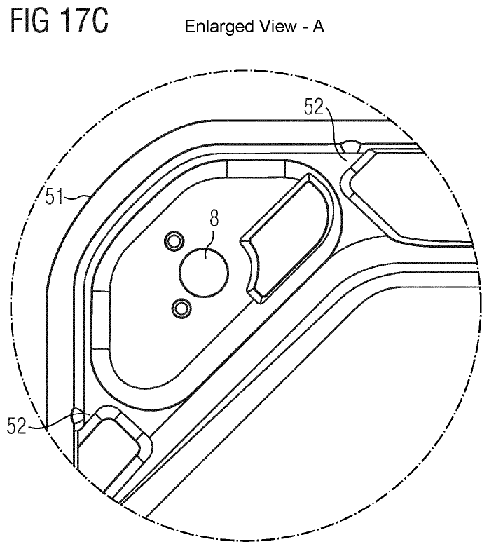

A further exemplary inlay 2 is shown in FIG. 17 in a perspective view (A) and in a top view (B). The section A from FIG. 17B is shown enlarged in FIG. 17C. Also this exemplary inlay 2 includes recesses 5, 6, 7 for receiving a tube. The recesses 5, 6 in the inlay 2 may not be fully closed at their sides (in a covered state), but may include a lateral cut-out 11, 12 at least one side of the recess 5, 6, such that one or more tubes may be laterally guided into and out of the recess 5, 6. Furthermore, the inlay may include a receiving section for an oxygenator (shown in FIGS. 2 and 3 with an oxygenator). The inlay 2 may furthermore include openings for a strap 16, 17. Furthermore, the inlay 2 may include openings for fastening a fixation clip 49. Advantageously, the inlay 2 includes an opening 8, through which the fastening means 3, 4 may be guided to fix the fastenings means 3, 4, e.g., in a rotatable manner, to the inlay 2, as described above. The inlay may advantageously include a respective opening 8 in diagonally opposing corners 50, 51 of the inlay 2, such that a respective fastening means 3, 4 may be arranged in the diagonally opposing corners 50, 51.

Furthermore, one corner 51 of the inlay 2 may advantageously include an arrow-shaped recess 52 (or an arrow-shaped elevation or protrusion), as shown in FIG. 17C, in order to further increase the stability of the inlay 2 in the tray 1. For this purpose, the tray 1, as shown in FIG. 18, may include an essentially form-fitting corresponding arrow-shaped recess 53 (or an arrow-shaped elevation) in the corresponding corner 32. The arrowhead of the arrow-shaped recess 52, 53 (or elevation) of the inlay 2 or of the tray 1 shows in a diagonal direction towards the corner 51, 32 (edge) of the inlay 2 or the tray 1. The tip or pinnacle of the arrowhead may be embodied as rounded or flattened (i.e. without an actual “spike”), as shown in FIGS. 17C and 18B. The rear, wide end of the arrowhead is arranged in an essentially perpendicular direction to the diagonal orientation of the arrow. Both edges of the recess (or elevation), which form the rear, wide end of the arrowhead, are hence arranged essentially perpendicular to the diagonal direction of the arrow. Accordingly, the inlay 2 may be fixed in the tray 1 even better. For this purpose it is particularly advantageous, if the inlay 2 and the tray 1, respectively, include two (form-fitting and between inlay 2 and tray 1 corresponding) arrow-shaped recesses 52, 53 (or elevations), in particular in diagonally opposing corners 50, 51, 31, 32 of the inlay 2 and tray 1, respectively. The diagonally opposing corners 50, 51 of the inlay 2 may be the same as those advantageously including an opening 8 for the fastening means 3, 4. Alternatively, these may also correspond to diagonally opposing corners, which do not include openings 8. It is particularly advantageous, if all four corners of the respective inlay 2 and tray 1 respectively include (form-fitting and between inlay 2 and tray 1 corresponding) arrow-shaped recesses 52, 53 (or elevations or protrusions). In this manner, it may be avoided that the inlay 2 slides or is moved within the tray 1, even in the case of a fall or drop (e.g. during transport).

An exemplary tubing set can easily be packed in the packaging system. First the medical tubing set is packed on inlay 2 outside tray 1; then the inlay 2 with the packed tubing set is placed in the tray 1; and then the inlay 2 (with the packed tubing set) is reversibly fastened in the tray 2.

To illustrate this packaging procedure, the packaging of an example tubing set including a blood tube 20, a gas conducting tube 21, an oxygenator 22, and a pump head 23 (and possibly various additional items that are usually used with the medical tubing set, such as tube clamps, a syringe 24, cable ties, etc.) in the example packaging system shown in FIGS. 1 to 18 is explained in more detail below. First, the fastening means 3, 4 is attached to the inlay 2. For this purpose, the base 4 of the fastening means can be brought into position (below the inlay 2 or the opening 8) in such a way that the handle 3 of the fastening means can be connected to the base 4 through the inlay 2 (i.e. through the opening 8) (e.g. by a snap-in function, e.g. of the extension 35 in the seat 36). In the packaging system shown as an example, this can be done twice, namely at diagonally opposite corners, to attach two fastening means to the inlay 2. A strap 17 is guided through openings provided for this purpose into the inlay 2. Covers 10 and 18 are equipped with the respective components intended for this purpose (e.g. additional items that are usually used together with the medical tubing set, such as tube clamps, a syringe 24, cable ties, etc.). The sequence of the above steps is arbitrary, since the inlay 2 and the covers 10 and 18 to be equipped can each be handled independently of each other. Then an oxygenator 22 can be inserted into the recess (provided for this purpose) or the receiving section of inlay 2. It may be advantageous to do this in such a way that the blood inlet or outlet port of the oxygenator 23 points to the right or the “short” gas line of the oxygenator 22 points to the left in the exemplary packaging system (see FIGS. 2 and 3). The oxygenator 22 can then be locked/secured to the inlay 2 with the strap 17. Then the tube ends of the tubing set can be guided around the oxygenator 22 (e.g. clockwise) and the tube may be rolled up in the recess 6 of the inlay 2. The open tube ends can be positioned on top of each other (e.g. with the respective tube clamp) in the recess 5 of the inlay 2. The tubes can then be locked in place with a partition 13, 14 (e.g. with two or three partitions 13, 14) in the corresponding cut-outs 11, 12 of the recess 5. The recess 6 (with the rolled-in tube) can then be covered with the cover 10, wherein the cover 10 can be locked to the inlay 2. If required, the pump head 23 can be aligned on the inlay 2, e.g. with the outer housing essentially parallel to the edge of inlay 2. By means of a fixing clip 49, the tubing can be locked above on the blood inlet side of the pump head 23 on the inlay 2, e.g. directly on the white spiral tubing. The cover 18 can be fixed (clamped) to the inlay 2 (or to the cover 10), e.g. so that the cover 18 is directly on the cover 10. Then a priming line can be inserted into inlay 2, e.g. on cover 18. In the packaging system shown here, the open tube ends may point to the left (to the oxygenator 22). The tube ends can be led or guided around the oxygenator 22 (e.g. counterclockwise). The open tube ends (Coulder couplings) can be inserted (e.g. with the respective tube clamps) one above the other in recess 5 of inlay 2. The tubes can then be locked in place with a partition 13, 14, e.g. one above the other, in the corresponding cut-out 11,12 of recess 5. Furthermore, further components (e.g. additional articles that are usually used together with the medical tubing set, such as a fir tree connector and Luer caps, etc.) can optionally be inserted in recess 5. Then the cover 9 can be locked on the inlay 2. On the cover 18 another tube, e.g. a (green) gas line (“long”) can be rolled up and deposited. The strap 16 can be guided through corresponding openings at the inlay 2. The cover 18 and the components inside it (e.g. a (green) gas line (“long”), a syringe 24, a priming line, etc.) are thus fixed to the inlay 2 by means of strap 17. The fully equipped inlay 2 is then placed in tray 1. The inlay 2 is fastened reversibly to the tray 1, e.g. by turning the handle 3 (or handles 3), e.g. clockwise by approx. 90.

After fastening the inlay 2 in the tray 1, it can be sealed, e.g., airtight, over the entire opening of tray 1. A foil (Topseal) can be used for this purpose. The (sealed) tray 1 can be packed in a secondary packaging (e.g. an outer carton). In such case the tray 1 can be put into a dust protection bag first and then be packed into a cardboard box, if necessary.

### LIST OF REFERENCE NUMERALS

1 Tray
2 Inlay
3 Upper part of the fastening means (handle)
4 Lower part of the fastening means (base)
5, 6, 7 Recess in the inlay for receiving a tube
8 Opening
9, 10 Cover for recess
11, 12 Lateral cut-out of the recess 5
13, 14 Partition
15 Recess in cover 10 for receiving a tube clamp
16, 17 Strap
18 Cover for (recesses of) further cover
19 Guide for strap 16
20 Tube for blood supply
21 Tube for gas supply
22 Oxygenator
23 Pump head
24 Syringe
25 Upper edge of the tray
26 Bottom of the tray
27 Support surface
28 Outer edge of the inlay
29 Receptacle for locking by the fastening element (fastening means)
30 Projection in (inner) wall of the tray
31, 32 Diagonally opposite corners of the tray
33 Wings in the lower part (e.g. base 4) of the fastening means
34 Gripping section of the (upper part of the) fastening means
35 Extension for connection to the lower part (base) of the fastening means
36 Seat for extension
37 Hook
38 Holder for hook
39 Connecting bar for integrating the hook into the fastening means
40-43 Opening in partition
44 End region of the strap with opening(s)
45 Openings in the end region 44 of the strap
46 End region 46 of the strap with a region (both sides) of reduced width and a widened region
47 Region (both sides) of reduced width (in the end region 46 of the strap)
48 Widened region (in the end region 46 of the strap)
49 Fixing clip
50,51 Diagonally opposing corners of the inlay
52 Arrow-shaped recess in corner of the inlay
53 Arrow-shaped recess in corner of the tray

1-24. (canceled)

25. A packaging system for a medical tubing set for extracorporeal therapy, the packaging system comprising:
an inlay with one or more recesses for accommodating components of a tubing set, and
a tray configured to receive the inlay and to reversibly fasten the inlay in the tray;
wherein a cavity is formed between the tray and the inlay.

26. The packaging system according to claim 25, wherein the packaging system is sterile.

27. The packaging system according to claim 25, wherein the inlay comprises recesses for receiving one or more tubes.

28. The packaging system according to claim 25, wherein the inlay comprises a receiving section for receiving an oxygenator.

29. The packaging system according to claim 25, wherein the inlay extends essentially over the entire area of the tray.

30. The packaging system according to claim 25, wherein the inlay is configured such that all components of the tubing set may be fully packaged on the inlay.

31. The packaging system according to claim 25, wherein the tray comprises a substantially horizontal support surface on its inner wall on which the inlay can be placed.

32. The packaging system according to claim 25, wherein the packaging system comprises a fastening device configured to reversibly fasten the inlay in the tray.

33. The packaging system according to claim 32, wherein the fastening device can reversibly fasten the inlay in the tray with a locking mechanism.

34. The packaging system according to claim 32, wherein the fastening device comprises a handle and a base, the handle comprising a gripping section and an extension for connection to the base, wherein the handle is configured to be passed through an opening in the inlay.

35. The packaging system according to claim 34, wherein the handle of the fastening device is connected to the inlay and is configured to serve for removal and/or transport of the inlay.

36. The packaging system according to claim 35, wherein the handle is configured to transport the inlay with components of the tubing set packaged thereon.

37. The packaging system according to claim 35, wherein the fastening device is rotatable, the base of the fastening device comprises a wing, and the inner wall of the tray comprises one or more projections which, depending on the position of the wing, cause the inlay to be fastened or detached in the tray.

38. The packaging system according to claim 37, wherein the wing is displaced relative to the one or more projections of the inner wall of the tray in such a way that fastening or detaching of the inlay in the tray is achieved when the fastening device is rotated.

39. The packaging system according to claim 25, wherein the packaging system further comprises a cover which at least partially covers a recess or a receiving section of the inlay.

40. The packaging system according to claim 39, wherein the cover partially covers a recess of the inlay, and wherein the recess in the inlay is not completely closed off laterally, but has a cut-out at least on one side of the recess, such that one or more tubes can be laterally inserted into the recess or extend laterally out of the recess.

41. The packaging system according to claim 40, wherein the packaging system further comprises a partition for form-fitting arrangement in the cut-out of the recess, wherein a tube can be passed through the partition.

42. The packaging system according to claim 39, wherein the packaging system comprises a cover for a recess of the inlay, wherein the cover comprises recesses for receiving objects for use with the medical tubing set.

43. The packaging system according to claim 42, wherein the objects for use with the medical tubing set comprise tubing clamps.

44. The packaging system according to claim 25, wherein the packaging system further comprises a strap for fixing one or more components of the tubing set.

45. The packaging system according to claim 44, wherein the packaging system further comprises a first cover for covering a recess of the inlay or for covering a second cover, wherein the first cover has a guide configured to receive the strap.

46. The packaging system according to claim 25, wherein the packaging system further comprises a fixing clip.

47. The packaging system according to claim 25, wherein the packaging system is configured to be unpacked in a predefined order.

48. A method of packaging a medical tubing set, the method comprising:
packaging the medical tubing set within a packaging system, the packaging system comprising:
an inlay with one or more recesses for accommodating components of the medical tubing set; and
a tray configured to receive the inlay and to reversibly fasten the inlay in the tray;
wherein a cavity is formed between the tray and the inlay.

49. A method of packaging a medical tubing set in a packaging system comprising an inlay with one or more recesses for accommodating components of the medical tubing set, and a tray configured to receive the inlay and to reversibly fasten the inlay in the tray, wherein a cavity is formed between the tray and the inlay, the method comprising:
packaging the medical tubing set on the inlay outside the tray;
inserting the inlay with the packaged tubing set into the tray; and
reversibly fastening the inlay in the tray by means of a fastening means.

50. The method according to claim 49, further comprising sealing the tray after fastening the inlay in the tray.

51. A fastening device for reversibly fastening an inlay in a tray, the fastening device comprising:
a handle and a base, the handle comprising a gripping section and an extension for connection to the base, wherein the handle is configured to be passed through an opening in the inlay;
wherein a hook is integrated in the handle, and wherein the hook can be released from the handle.
